(19) United States
(12) Patent Application Publication
(10) Pub. No.: US 2025/0357013 A1
(43) Pub. Date: Nov. 20, 2025

(54) SYSTEMS FOR FACILITATING PATIENT INTERACTIONS

(71) Applicant: BAYLOR RESEARCH INSTITUTE D/B/A BAYLOR SCOTT & WHITE RESEARCH INSTITUTE, Dallas, TX (US)

(72) Inventors: Crystal Sokolowski, Jackson, TN (US); Jordan Hanson, Temple, TX (US); Adan Torres, JR., Belton, TX (US); John Patton, Temple, TX (US); Marlene Porter, Temple, TX (US)

(21) Appl. No.: 19/213,259

(22) Filed: May 20, 2025

Related U.S. Application Data
(60) Provisional application No. 63/649,674, filed on May 20, 2024.

(51) Int. Cl. G16H 80/00; G06F 3/01; G06F 3/04812; G06F 3/04817; G06F 3/0482; G06F 3/0485; G06F 3/04886; G09B 21/00

(52) U.S. Cl. CPC G16H 80/00; G06F 3/012; G06F 3/013; G06F 3/017; G06F 3/04812; G06F 3/04817; G06F 3/0482; G06F 3/0485; G06F 3/04886; G09B 21/00

(57) ABSTRACT

A system can comprise an electronic device configured to display, on an electronic display thereof, one or more communication pages that include a plurality of icons that comprise a plurality of communication icons. The electronic device can be configured to determine which of the icons is a selected icon based at least in part on a communication input, and can produce a sound when the selected icon is one of the communication icons. The electronic device can be configured to determine, based at least on part on sensorimotor inputs, which of two or more communications modes is a recommended communication mode, wherein for each of the communication modes, the communication input is different when the electronic device is in the communication mode than when the electronic device is in at least one other of the communication modes.

### CROSS-REFERENCE TO RELATED APPLICATIONS

This application claims the benefit of priority of U.S. Provisional Patent Application No. 63/649,674, filed May 20, 2024, which is hereby incorporated by reference in its entirety.

### FIELD OF INVENTION

The present invention relates generally to systems and methods for facilitating patient interactions, as well as to medical carts.

### BACKGROUND

In the healthcare setting, communication between a patient and others—such as a caregiver, family member, or friend—can be important to, for example, allow the patient to convey information about their condition and needs. While many patients can communicate with others through speech, some may not be able to do so. For example, some patients may have an injury, suffer from a medical condition, and/or be outfitted with one or more medical devices that prevent them from speaking. And some patients may only be able to speak in a language that another individual like a caregiver cannot understand. Patients unable to communicate with others via speech may have limited options for conveying information about their condition and what they need, which can impair a caregiver's ability to take care of the patient. While some patients may be able to transcribe what they wish to communicate in handwriting or by typing, doing so can be inefficient and cumbersome, and some patients with limited use of their fingers and hands may not be able to do so. Thus, communicating with patients who cannot speak with a caregiver remains a challenge in the healthcare setting.

### SUMMARY

Embodiments of the present systems can include an electronic device that a patient can use to efficiently communicate with another individual such as a caregiver even though the patient cannot speak or can only speak in a language that the other individual cannot understand. The electronic device can comprise an electronic display and can be configured to display, on the electronic display, one or more communication pages that each include a plurality icons that comprise a plurality of communication icons. Based at least in part on a communication input, the electronic device can be configured to determine which of the icons is a selected icon, and when one of the communication icons is the selected icon, the electronic device can produce, via one or more speakers thereof, a sound based at least in part on which of the communication icons is the selected icon. The sound can comprise, for example, one or more words that reflect what is meant to be communicated with the selection of the particular communication icon. The communication icons can include ones that, when selected, cause the electronic device to produce sound (e.g., one or more words) that allows the patient to convey information relevant to the patient's care in a healthcare facility, such as pain, feelings, room and/or bed adjustments, sanitary needs, bathroom needs, and/or the like. The selection of icons on the communication page(s) can thus allow a voiceless patient to communicate with others in a meaningful way.

To accommodate patients having different sensorimotor abilities, the electronic device can have multiple communication modes, where for each of the communication modes the communication input used to select one of icons is different when the electronic device is in the communication mode than when the electronic device is in at least one other of the communication modes. In this manner, a patient that may not be able to make the communication input of one of the communication modes may nevertheless be able to make the communication input of another one of the communication modes to select the icons and accordingly communicate with another individual.

The electronic device can be configured to determine which of the communication modes is the recommended communication mode for the patient and accordingly facilitate the use of a communication mode that can allow a patient to reliably and efficiently select icons. To do so, the electronic device can be configured to display, on the electronic display, at least one or more sensorimotor assessment pages that include a plurality of prompts, wherein each of the prompts requests a sensorimotor input that comprises a selection of one of a plurality of options regarding a sensorimotor ability of the patient. The determination of which of the communication modes is the recommended communication mode can be based at least in part on the sensorimotor inputs.

For example, if the electronic display is a touchscreen, touching the touchscreen may be the most reliable way of selecting one of icons on the communication page(s). The sensorimotor prompts can thus include a touch prompt requesting a sensorimotor input regarding whether the patient can touch the touchscreen with one of their fingers, and if the sensorimotor input indicates that the patient can do so, the electronic device can determine that a touch-based communication mode is the recommended communication mode. If the sensorimotor input requested by the touch prompt indicates that the patient cannot touch the touchscreen, the electronic device can be configured to determine that another one of the communication modes that does not require the patient to touch the touchscreen is the recommended communication mode.

To illustrate, the communication modes can include one or more cursor-based communication modes in which one or more cursors can move on a communication page and the patient can make an icon selection input-such as a facial gesture or a sound that is not a word—to select the icon on which the cursor(s) are disposed. Such cursor-based communication mode(s) can include one or more head-based communication modes in which the patient's head movements can be detected by the electronic device (e.g., using a camera) to move the cursor(s) and/or one or more eye-based communication modes in which the patient's eye movements can be detected by the electronic device (e.g., using the camera) to move the cursor(s), allowing the patient to make selections without the use of their hands.

To further illustrate, the communication modes can include one or more scroll-based communication modes in which the electronic device is configured to display an indication regarding which one of the icons is a selectable icon, where when the icon selection input is made, the electronic device can be configured to determine that the selectable icon is the selected icon. Which of the icons is the selectable icon can change when the patient makes a switching input or automatically after a threshold selection time. Such scroll-based communication mode(s) can be other alternatives that allow patients with sensorimotor impediments to use the electronic device, especially patients that cannot move their head or eyes and thus would not be able to select icons if the electronic device was in one of the head-based or eye-based communication modes.

The electronic device can be configured to determine which of the communication modes is a recommended communication mode in a manner that, subject to the patient's abilities, prioritizes communication modes in which the patient can efficiently make and the electronic device can more-reliably detect inputs, which can allow the electronic device to more consistently be used in a mode that best facilitates the patient's use thereof, compared to, for example, a purely manual configuration of the electronic device. As described in further detail below, depending at least in part on inputs regarding the patient's abilities, the electronic device can, for example, prioritize touch-based, head-based, and eye-based communication modes (e.g., in that order) over others like the scroll-based communication modes, where patient impediments can indicate that otherwise less-preferred communication modes may be better suited for the patient.

Some of the present systems for facilitating patient interactions comprise an electronic device. The electronic device, in some embodiments, comprises one or more processors, an electronic display, and/or one or more speakers.

In some embodiments, the electronic device is configured to display, on the electronic display, one or more sensorimotor assessment pages that include a plurality of prompts. Each of the prompts of the sensorimotor assessment page(s), in some embodiments, comprises a selection of one of a plurality of options regarding a sensorimotor ability of a patient.

In some embodiments, the electronic device is configured to determine, based at least in part on the sensorimotor inputs, which of two or more communication modes of the electronic device is a recommended communication mode. The electronic device, in some embodiments, is configured to automatically enter into the recommended communication mode and/or display, on the electronic display, which of the communication modes is the recommended communication mode.

In some embodiments, the electronic device is configured to display, on the electronic display, one or more communication pages. The communication page(s), in some embodiments, include a plurality of icons that comprise a plurality of communication icons.

In some embodiments, the electronic device is configured to determine, based at least in part on a communication input, which of the icons of the communication page(s) is a selected icon. In some embodiments, for each of the communication modes, the communication input on which the determination is at least in part based is different when the electronic device is in the communication mode than when the electronic device is in at least one other of the communication modes.

The communication modes, in some embodiments, comprise one or more cursor-based communication modes. In some embodiments, for each of the cursor-based communication mode(s), when the electronic device is in the cursor-based communication mode, for each of the communication page(s), the electronic device is configured to display, on the electronic display, one or more cursors on the communication page. In some of such embodiments, when the electronic device is in the cursor-based communication mode, the communication input comprises an icon selection input that includes a facial gesture, a sound, and/or maintaining the cursor(s) within an area on one of the communication page(s) for greater than or equal to a threshold dwell time. In some of such embodiments, the electronic device is configured such that, when the cursor(s) are disposed over one of the icons of the communication page(s) and the icon selection input is made, the electronic device determines that the icon over which the cursor(s) are disposed is the selected icon.

In some embodiments, the cursor-based communication mode(s) include one or more head-based communication modes. In some embodiments, for each of the head-based communication mode(s), when the electronic device is in the head-based communication mode, the communication input comprises movement of a head of the patient and, for each of the communication page(s), the electronic device is configured to determine a position of the cursor(s) on the communication page based at least in part on a position of the head of the patient.

In some embodiments, the cursor-based communication mode(s) include one or more eye-based communication modes. In some embodiments, for each of the eye-based communication mode(s), when the electronic device is in the eye-based communication mode, the communication input comprises movement of at least one of one or more eyes of the patient and, for each of the communication page(s), the electronic device is configured to determine a position of the cursor(s) on the communication page based at least in part on a position of at least one of the eye(s) of the patient.

In some embodiments, the cursor-based communication mode(s) comprise one or more gliding-cursor-based communication modes. For each of the gliding-cursor-based communication mode(s), in some embodiments, when the electronic device is in the gliding-cursor-based communication mode, the one or more cursors comprise first and second linear cursors. The first linear cursor, in some of such embodiments, extends in a first direction and the second linear cursor, in some of such embodiments, extends in a second direction that is substantially perpendicular to the first direction. In some of such embodiments, the communication input comprises a stop input. The stop input, in some embodiments, includes a facial gesture and/or a sound. In some embodiments, when the electronic device is in the gliding-cursor-based communication mode, for each of the communication page(s), the electronic device is configured to display, on the electronic display, the first linear cursor moving in the second direction, wherein the electronic device is configured to stop the first linear cursor and display the first linear cursor at a position at which the first linear cursor is stopped when the stop input is made, and after the first linear cursor is stopped, the second linear cursor moving in the first direction. In some of such embodiments, when an intersection point at which the first linear cursor intersects the second linear cursor is disposed over one of the icons of the communication page(s) and the icon selection input is made, the electronic device is configured to determine that the icon over which the intersection point is disposed is the selected icon.

The communication modes, in some embodiments, include one or more scroll-based communication modes. In some embodiments, for each of the scroll-based communication mode(s), when the electronic device is in the scroll-based communication mode, the electronic device is configured to determine which one of the icons of the communication page(s) is a selectable icon and display, on the electronic display, an indication regarding which one of the icons of the communication page(s) is the selectable icon. In some of such embodiments, the communication input comprises an icon selection input that includes a facial gesture and/or a sound. In some of such embodiments, the electronic device is configured such that, for each of the icons of the communication page(s), when the icon is the selectable icon and the icon selection input is made, the electronic device determine that the icon is the selected icon.

In some embodiments, the scroll-based communication mode(s) comprise one or more switching-based communication modes. For each of the switching-based communication mode(s), in some embodiments, when the electronic device is in the switching-based communication mode, the communication input comprises a switching input. The switching input, in some embodiments, comprises a facial gesture and/or a sound. In some embodiments, the switching input is different than the icon selection input. In some embodiments, for each of the icons of the communication page(s), when the icon is the selectable icon and the switching input is made, the electronic device is configured to determine that the icon is no longer the selectable icon and that another one of the icons is the selectable icon.

In some embodiments, the scroll-based communication mode(s) comprise one or more gliding-switch-based communication modes. For each of the gliding-switch-based communication mode(s), in some embodiments, when the electronic device is in the gliding-switch-based communication mode, for each of the icons of the communication page(s), the electronic device is configured such that when the icon has been the selectable icon for a threshold selection time, the electronic device determines that the icon is no longer the selectable icon and that another one of the icons is the selectable icon.

In some embodiments, the electronic display of the electronic device comprises a touchscreen and the communication modes of the electronic device comprise one or more touch-based communication modes. In some of such embodiments, for each of the touch-based communication mode(s), when the electronic device is in the touch-based communication mode, the communication input comprises an icon selection input that includes touching the touchscreen. In some embodiments, the electronic device is configured such that when the touchscreen is touched at a location disposed over one of the icons of the communication page(s), the electronic device determines that the icon over which the touched location is disposed is the selected icon.

In some embodiments, the prompts of the sensorimotor assessment page(s) comprise a touch prompt, wherein the sensorimotor input that the touch prompt requests comprises a selection of one of a plurality of options regarding whether the patient can touch the touchscreen with one of a plurality of fingers of the patient. In some of such embodiments, the electronic device is configured such that when determining which of the communication modes is the recommended communication mode, when the sensorimotor input that the touch prompt requests indicates that the patient can touch the touchscreen with one of the fingers of the patient, one of the touch-based communication mode(s) is determined to be the recommended communication mode.

In some embodiments, the prompts of the sensorimotor assessment page(s) comprise a head-movement prompt. The sensorimotor input that the head-movement prompt requests, in some embodiments, comprises a selection of one of a plurality of options regarding whether the patient can move their head. In some of such embodiments, the electronic device is configured such that when determining which of the communication modes is the recommended communication mode, when the sensorimotor input that the head-movement prompt requests indicates that the patient can move their head, none of the eye-based communication mode(s) are determined to be the recommended communication mode. In some embodiments, the electronic device is configured such that when determining which of the communication modes is the recommended communication mode, when the sensorimotor input that the head-movement prompt requests indicates that the patient can move their head, none of the scroll-based communication mode(s) are determined to be the recommended communication mode.

In some embodiments, the prompts of the sensorimotor assessment page(s) comprise an eye-movement prompt. The sensorimotor input that the eye-movement prompt requests, in some embodiments, comprises a selection of one of a plurality of options regarding whether the patient can move their eye(s). In some of such embodiments, the electronic device is configured such that when determining which of the communication modes is the recommended communication mode, when the sensorimotor input that the eye-movement prompt requests indicates that the patient can move their eye(s) and the electronic device determines that it can track movement of the eye(s) of the patient and the patient is capable of moving the cursor(s) by moving their eye(s), one or more of the scroll-based communication mode(s) are not determined to be the recommended communication mode.

In some embodiments, the electronic device is configured to display, on the electronic display, one or more consciousness assessment pages that include a prompt that requests a consciousness input. The consciousness input, in some embodiments, comprises a selection of one of a plurality of options regarding a level of consciousness of the patient. In some embodiments, the options include a first level of consciousness and a second level of consciousness that is lower than the first level of consciousness. In some of such embodiments, the electronic device is configured such that when determining which of the communication modes is the recommended communication mode, when the consciousness input indicates that the patient has the second level of consciousness, one of the gliding-cursor-based communication mode(s) is determined to be the recommended communication mode. In some embodiments, the electronic device is configured such that when determining which of the communication modes is the recommended communication mode, when the consciousness input indicates that the patient has the second level of consciousness, one of the gliding-switch-based communication mode(s) is determined to be the recommended communication mode.

In some embodiments, the electronic device is configured to determine whether the patient is capable of making at least one of one or more facial gestures. In some embodiments, the prompts of the sensorimotor assessment page(s) comprise a facial-gesture prompt, wherein the sensorimotor input that the facial-gesture prompt requests comprises a selection of one of a plurality of options regarding whether the patient can make a facial gesture. In some embodiments, the prompts of the sensorimotor assessment page(s) comprise a sound prompt, wherein the sensorimotor input that the sound prompt requests comprises a selection of one of a plurality options regarding whether the patient can make a sound with their mouth and/or tongue. For each of the cursor-based communication mode(s) and/or scroll-based communication mode(s), in some embodiments, the electronic device is configured to determine which one of two or more types of selection inputs is the icon selection input. The types of selection inputs, in some embodiments, include one or more facial gestures and/or one or more sounds. In some embodiments, the electronic device is configured such that when determining which of the types of selection inputs is the icon selection input, when the electronic device determines that the patient is capable of making at least one of one or more facial gestures, none of the sound(s) of the types of selection inputs are determined to be the icon selection input.

In some embodiments, the electronic device is configured to determine whether the patient is capable of making one or more lower facial gestures that are each from a portion of a face of the patient that is below a nose of the patient and one or more upper facial gestures that are each from a portion of the face of the patient that is at or above the nose of the patient. The lower facial gesture(s), in some embodiments, comprise poking out a tongue, opening a mouth, smiling, and/or grimacing. In some embodiments, for each of the cursor-based communication mode(s) and/or scroll-based communication mode(s), the electronic device is configured to determine which one of two or more types of selection inputs is the icon selection input, the types of selection inputs including the lower facial gesture(s) and the upper facial gesture(s). The electronic device, in some embodiments, is configured such that when determining which of the types of selection inputs is the icon selection input, when the electronic device determines that the patient is capable of making at least one of the lower facial gesture(s), none of the upper facial gesture(s) are determined to be the icon selection input.

In some embodiments, the electronic device is configured to display, on the electronic display, one or more device assessment pages that include a prompt that requests a device input, the device input comprising a selection of at least one of a plurality of options regarding whether the patient is outfitted with one or more devices. For at least one of the sensorimotor inputs, in some embodiments, the electronic device is configured to preselect, based at least in part on the device input, one of the options regarding the sensorimotor ability of the patient. The one or more devices, in some embodiments, comprise one or more immobility devices configured to inhibit movement of the head of the patient. In some of such embodiments, the electronic device is configured such that when the device input indicates that the patient is outfitted with at least one of the immobility device(s), the electronic device preselects one of the options for the head-movement prompt indicating that the patient cannot move their head. In some embodiments, the one or more devices comprise one or more mouth devices configured to be disposed on and/or through the mouth of the patient. In some of such embodiments, the electronic device is configured such that when the device input indicates that the patient is outfitted with at least one of the mouth device(s), the electronic device preselects one of the options for the facial-gesture prompt indicating that the patient cannot make a facial gesture and one of the options for the sound prompt indicating that the patient cannot make a sound with their mouth and/or tongue.

In some embodiments, the prompts of the sensorimotor assessment page(s) comprise a visual-acuity prompt. In some of such embodiments, the sensorimotor input that the visual-acuity prompt requests comprises a selection of one of a plurality of options regarding a visual acuity of the patient. The options of the sensorimotor input of the visual-acuity prompt, in some embodiments, include a first level of visual acuity and a second level of visual acuity that is lower than first level of visual acuity. In some of such embodiments, for each of the communication modes of a first portion of the communication modes of the electronic device, when the electronic device is in the communication mode, each of the communication icons has a first area, and for each of the communication modes of a second portion of the communication modes of the electronic device, when the electronic device is in the communication mode, each of the communication icons has a second area that is larger than the first area. In some embodiments, the electronic device is configured to determine that one of the communication modes of the second portion of the communication modes is the recommended communication mode when the sensorimotor input that the visual-acuity prompt requests indicates that the patient has the second level of visual acuity.

In some embodiments, the electronic device is configured to produce, via the speaker(s) a sound when the selected icon is one of the communication icons. The sound, in some embodiments, is based at least in part on which of the communication icons is the selected icon.

In some embodiments, the electronic device is configured to display, on the electronic display, one or more language assessment pages. The language assessment page(s), in some embodiments, include a caregiver language prompt that requests a caregiver language input that comprises a selection of one of a plurality of options regarding a language that a caregiver is able to understand and/or a patient language prompt that requests a patient language input that comprises a selection of one of a plurality of options regarding a language that the patient is able to understand. In some of such embodiments, each of the communication icons includes a marking in the language that the patient language input indicates the patient is able to understand. In some embodiments, the sound that the electronic device is configured to produce based at least in part on which of the communication icons is the selected icon includes a sound in the language that the caregiver language input indicates the caregiver is able to understand.

Some systems comprise a medical cart. The medical cart, in some embodiments, includes a base. The base, in some embodiments, includes a central portion. In some embodiments, the base includes a plurality of supporting legs. The supporting legs, in some embodiments, each extend at least radially outward from the central portion. In some embodiments, the base includes a plurality of wheels, each coupled to a respective one of the supporting legs. Each of the wheels, in some embodiments, is disposed closer to an outer end of the supporting leg to which it is coupled than to the central portion. The cart, in some embodiments, includes a post. The post, in some embodiments, is coupled to and extends upwardly away from the central portion of the base. In some embodiments, the electronic device is coupled to the post such that the electronic device is disposed higher than the base.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified—and includes what is specified, e.g., substantially 90 degrees includes 90 degrees and substantially parallel includes parallel—as understood by a person of ordinary skill in the art. As used herein, “substantially parallel” means within 10 degrees of parallel to, and “substantially perpendicular” means within 10 degrees of perpendicular to.

The terms “comprise” and any form thereof such as “comprises” and “comprising,” “have” and any form thereof such as “has” and “having,” and “include” and any form thereof such as “includes” and “including” are open-ended linking verbs. As a result, an apparatus or system that “comprises,” “has,” or “includes” one or more elements possesses those one or more elements but is not limited to possessing only those elements. Likewise, a method that “comprises,” “has,” or “includes” one or more steps possesses those one or more steps but is not limited to possessing only those one or more steps.

Any embodiment of any of the apparatuses, systems, and methods can consist of or consist essentially of—rather than comprise/have/include-any of the described steps, elements, and/or features. Thus, in any of the claims, the term “consisting of” or “consisting essentially of” can be substituted for any of the open-ended linking verbs recited above in order to change the scope of a given claim from what it would otherwise be using the open-ended linking verb.

Further, an apparatus or system that is configured in a certain way is configured in at least that way, but it can also be configured in other ways than those specifically described.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 is a perspective view of a first embodiment of the present systems for facilitating patient interactions that comprises an electronic device and a medical cart.

FIGS. 2A-2C are front, bottom, and top views, respectively, of the electronic device of the system of FIG. 1.

FIG. 3A is a front view of the electronic device of FIGS. 2A-2C when displaying a first one of a plurality of communication pages that include a plurality of icons, wherein at least some of the icons include communication icons that, when selected by a patient, can cause the electronic device to produce a sound.

FIG. 3B is a front view of the electronic device of FIGS. 2A-2C when displaying a second one of the communication pages over the first communication page, the second communication page including a plurality of the communication icons regarding the level and description of pain the patient is experiencing.

FIG. 3C is a front view of the electronic device of FIGS. 2A-2C when displaying a third one of the communication pages over the first communication page, the third communication page allowing a patient to indicate where they are experiencing pain.

FIG. 3D is a front view of the electronic device of FIGS. 2A-2C when displaying a fourth one of the communication pages over the first communication page, the fourth communication page including a plurality of the communication icons regarding how the patient is feeling.

FIG. 3E is a front view of the electronic device of FIGS. 2A-2C when displaying a text-entry page in which the patient can enter a text string that the electronic device can recite.

FIG. 4 is a front view of the electronic device of FIGS. 2A-2C when displaying the first communication page and when the electronic device is in one of a plurality of cursor-based communication modes.

FIGS. 5A-5C are a front views of the electronic device of FIGS. 2A-2C when displaying the first communication page and when the electronic device is in a gliding-cursor-based communication mode. In FIG. 5A, a first linear cursor moving in a first direction is displayed on the communication page, in FIG. 5B a second linear cursor moving in a second direction is displayed on the communication page while the first linear cursor is stopped, and in FIG. 5C the first and second linear cursors are stopped and intersect over one of the communication icons.

FIG. 6 is a front view of the electronic device of FIGS. 2A-2C when displaying the first communication page and when the electronic device is in one of a plurality of scroll-based communication modes.

FIG. 7 is a block diagram of an algorithm implemented by the electronic device of FIGS. 2A-2C to configure the electronic device and determine which of the communication modes is a recommended communication mode.

FIG. 8A is a front view of the electronic device of FIGS. 2A-2C when displaying a language assessment page that includes prompts for languages that the caregiver and patient are able to understand.

FIG. 8B illustrates one of the communication icons and how the inputs requested on the language assessment page can affect the communication icons.

FIG. 9 is a front view of the electronic device of FIGS. 2A-2C when displaying an avatar assessment page including a prompt for the patient's gender.

FIG. 10 is a front view of the electronic device of FIGS. 2A-2C when displaying an education assessment page including a prompt regarding whether the patient has previously used the device.

FIG. 11 is a front view of the electronic device of FIGS. 2A-2C when displaying a consciousness assessment page that includes a prompt regarding a level of consciousness of the patient.

FIG. 12 is a front view of the electronic device of FIGS. 2A-2C when displaying a device assessment page that includes a prompt regarding what, if any, devices the patient is outfitted with.

FIG. 13 is a front view of the electronic device of FIGS. 2A-2C when displaying a sensorimotor assessment page that includes a plurality of prompts regarding the patient's sensorimotor abilities.

FIG. 14 is a block diagram of an algorithm implemented on the electronic device of FIGS. 2A-2C for determining which of the communication modes is the recommended communication mode based at least in part on one or more inputs requested on previous assessment pages.

FIG. 15A is a block diagram of an algorithm implemented on the electronic device of FIGS. 2A-2C for determining which touch-based communication mode is the recommended communication mode.

FIG. 15B is a front view of the electronic device of FIGS. 2A-2C when displaying the first communication page and when the electronic device is in a touch-based communication mode employing enlarged communication icons.

FIG. 16A is a block diagram of an algorithm implemented on the electronic device of FIGS. 2A-2C for determining which head-based or eye-based communication mode is recommended, which includes determining whether to use a facial gesture, dwell time, or a sound as an icon selection input.

FIG. 16B is a block diagram of an algorithm implemented on the electronic device of FIGS. 2A-2C for determining which of a plurality of facial gestures to use as the icon selection input for the head-based or eye-based communication modes.

FIG. 16C is a block diagram of an algorithm implemented on the electronic device of FIGS. 2A-2C for determining which of a plurality of sounds to use as the icon selection input for the head-based or eye-based communication modes.

FIG. 17 is a block diagram of an algorithm implemented on the electronic device of FIGS. 2A-2C for determining which switching-based communication mode is the recommended communication mode.

FIG. 18 is a block diagram of an algorithm implemented on the electronic device of FIGS. 2A-2C for determining which gliding-based communication mode is the recommended communication mode, which includes determining whether to use a facial gesture or a sound as an icon selection input.

FIG. 19 is a front view of the electronic display of FIGS. 2A-2C displaying a scene-based communication page.

FIGS. 20A and 20B are front and top views, respectively, of the system of FIG. 1 while the electronic device is in a deployed position.

FIG. 21 is a top view of the system of FIG. 1 while the electronic device is in a stowed position for transportation.

FIG. 22 is a partial front view of a second embodiment of the present systems in which the cart comprises one or more motors configured to move the electronic device.

FIGS. 23A and 23B depict some of the present methods of using the system of FIG. 1 in which the cart is rolled to a bed on which a patient is disposed (FIG. 23A) and the electronic device is moved to the deployed position (FIG. 23B).

### DETAILED DESCRIPTION

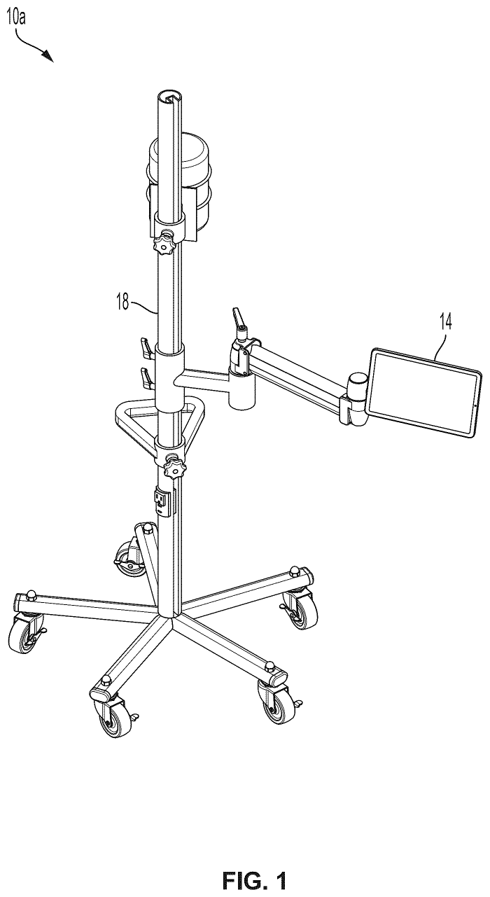

Referring to FIG. 1, shown is a first embodiment 10a of the present systems for facilitating patient interactions. System 10a can comprise an electronic device 14 that can be configured to help a patient communicate with another individual-like a caregiver, a family member, or a friend-when the patient might not otherwise be able to communicate with them, such as when the patient cannot speak or does not speak a language that the other individual can understand. Such communications can allow the patient to, for example, make requests or inform the other individual about how the patient feels physically and/or emotionally, which can facilitate the care of the patient. Furthermore, system 10a can comprise a cart 18 that electronic device 14 can be coupled to such that the electronic device can be readily transported in, for example, a healthcare facility and can be supported while in use.

### A. Electronic Device

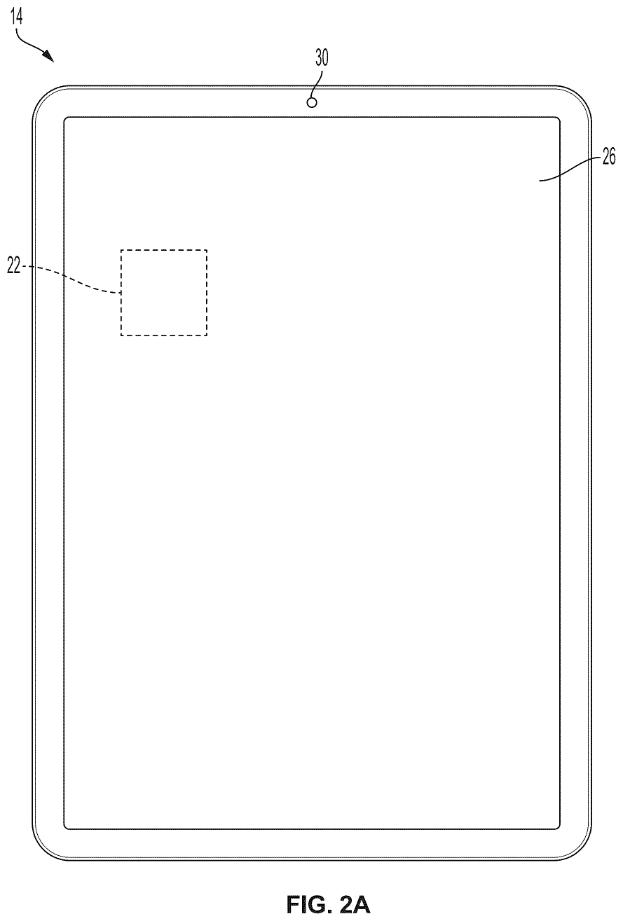

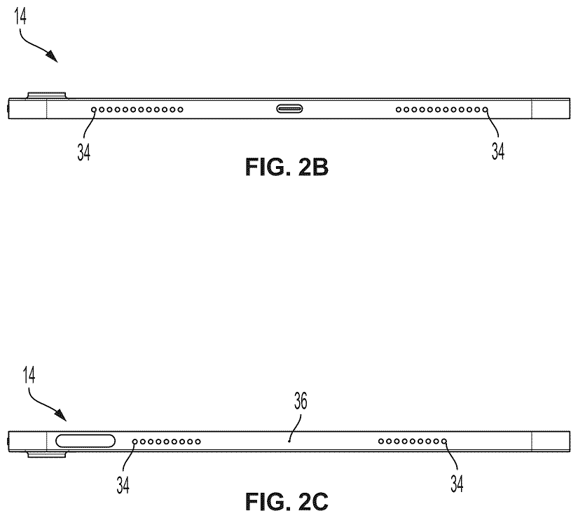

Referring to FIGS. 2A-2C, electronic device 14 can comprise any suitable electronic device that can run the below-described program to facilitate patient interactions, such as a tablet, a computer like a laptop, and/or the like. Preferably, electronic device 14 is a portable electronic device such as a tablet (e.g., as shown) such that the electronic device can be easily handled and maneuvered for use by a patient. To run such a program, electronic device 14 can comprise one or more processors 22 that can be configured to execute machine-readable instructions stored on a non-transitory storage medium of the electronic device and an electronic display 26 that can display one or more graphical user interfaces for a user. Additionally, electronic device 14 can comprise a camera 30 configured to face a patient when the user is looking at electronic display 26 such that the camera can observe the patient's head, face, and/or eye movements, a microphone 36 that can detect sounds made by the patient, and one or more speakers 34, wherein at least one (up to and including each) of the camera, microphone, and speaker(s) can be housed in a housing of the electronic device (e.g., as shown) or can be external to the housing. As explained in further detail below, the patient can use such movements and sounds to select what the patient wishes to communicate to another individual, and electronic device 14's speaker(s) 34 can produce sounds conveying the selected communication to the other individual.

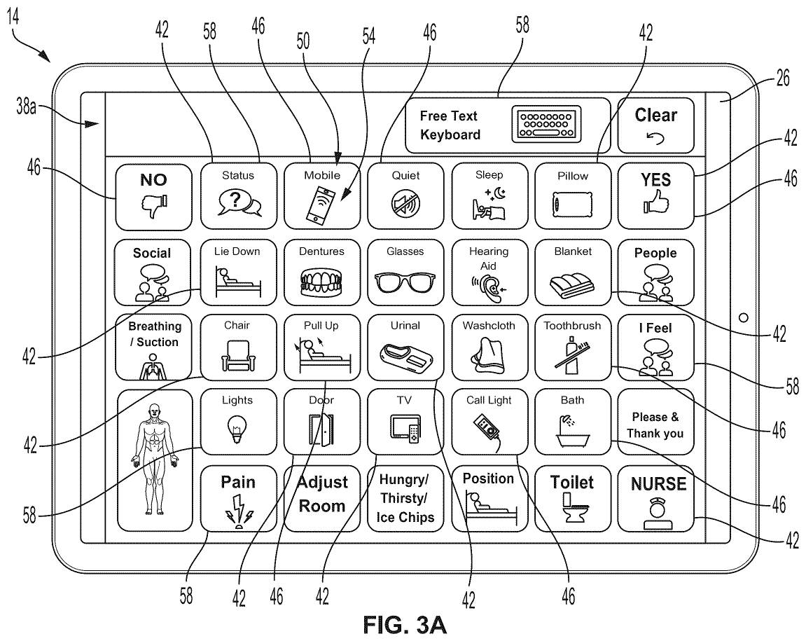

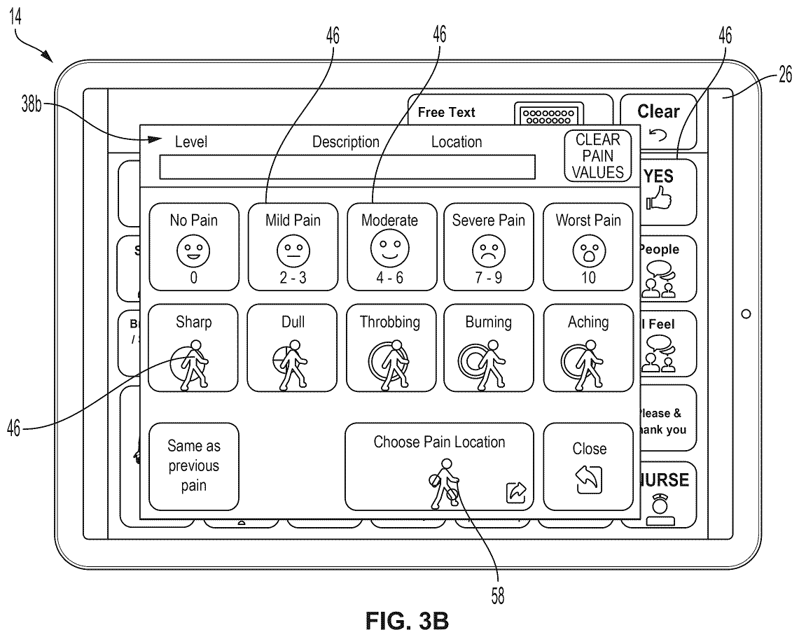

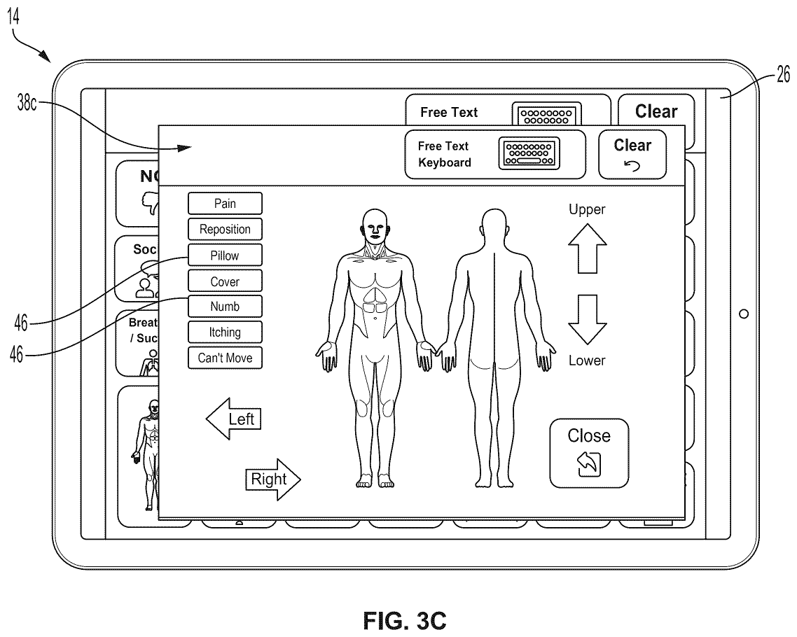

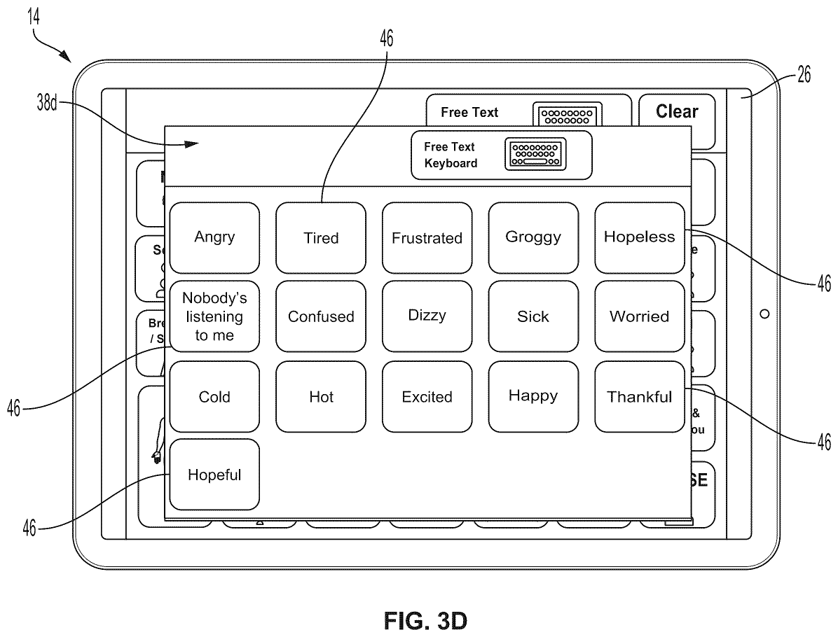

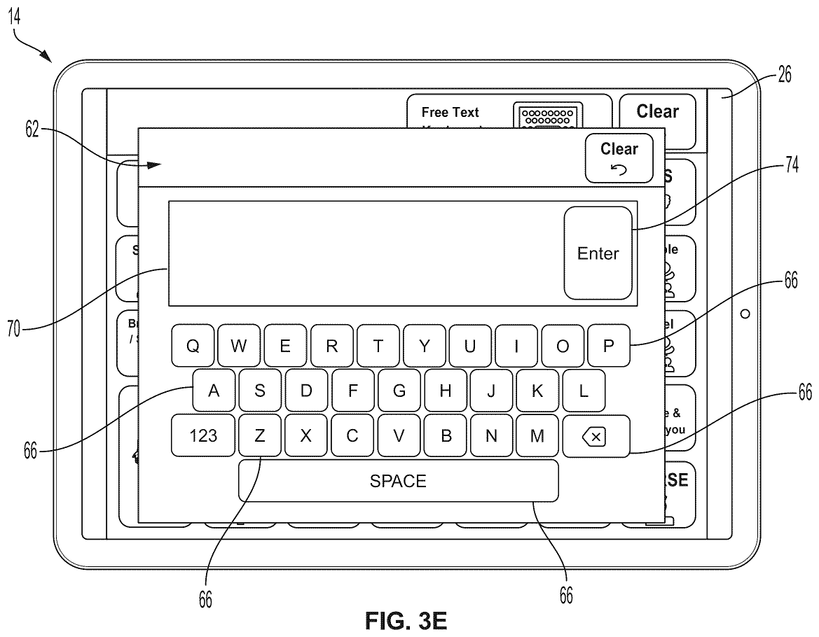

Turning to FIGS. 3A-3E, to allow a patient that cannot speak or does not speak a language understandable by the other individual (e.g., a caregiver) to communicate with them, electronic device 14 can be configured to display, on electronic display 26, one or more communication pages 38a-38d that the patient can interact with to achieve the desired communication. Communication page(s) 38a-38d can include a plurality of icons 42 that the patient can select, where each of the icons can include a marking 50 in a language that the patient understands and/or a logo 54 that are indicative of what electronic device 14 can communicate with selection of the icon.

Icons 42 of communication page(s) 38a-38d can include a plurality of communication icons 46, where when the patient selects one of the communication icons, electronic device 14 can produce, via its speaker(s) 34, a sound that is based at least in part on which of the communication icons is the selected icon. The sound that electronic device 14 can produce when one of communication icons 46 is selected can include one or more words that communicate what the communication icon's marking 50 and/or logo 54 indicate. To illustrate, as shown in FIG. 3A, communication icons 46 of a first communication page 38a can include “NO” and “YES” communication icons that, when selected, can cause electronic device 14 to produce, via speaker(s) 34, a sound stating “No” or “Yes,” respectively, allowing the patient to respond to Yes/No questions posited by the individual with which the patient is communicating. As another example, the selection of “Pull Up” communication icon 46 can cause electronic device 14 to a produce a sound requesting the patient to be positioned higher in the bed (e.g., “Please pull me higher in the bed.”). Additionally or alternatively, electronic device 14 can be configured to communicate with a remote electronic device (e.g., over one or more networks) such that, when one of communication icons 46 is selected, the electronic device can communicate with the remote electronic device to cause the remote electronic device to produce, via one or more speakers of the remote electronic device, a sound that is based at least in part on which of the communication icons is the selected icon. The sound that the remote electronic device can produce can be the same that electronic device 14 is configured to produce (e.g., one or more words that communicate what the selected communication icon 46's marking 50 and/or logo 54 indicate). In this manner, the patient can also use electronic device 14 to communicate with another disposed at a remote location, which can be particularly beneficial for those who lack the mobility and dexterity to use, for example, a mobile phone. To facilitate communication with such a remote user, electronic device 14 can be configured to produce, via its speaker(s) 34, sound captured by the remote electronic device (e.g., words spoken by the remote user) and/or display, on electronic display 26, video captured by the remote electronic device (e.g., of the remote user) so that the patient can hear and/or see the remote user.

Icons 42 can also include one or more-optionally a plurality of—expansion icons 58, wherein when one of communication page(s) 38a-38d is displayed and one of the expansion icon(s) that is on the communication page is selected, electronic device 14 can be configured to display, on electronic display 26, another page, such as (when there are multiple communication pages) another one of the communication pages that includes at least one different communication icon. This can allow the patient access to more communication icons 46 than what may be readably displayable on a single page on electronic display 26 and can allow better organization of the communication icons to facilitate the patient's ability to find a communication icon for what the patient wishes to communicate. For at least one of expansion icon(s) 58, electronic device 14 can also be configured to produce a sound, via its speaker(s) 34, when the expansion icon is selected; the sound produced can comprise, for example, one or more words conveying at least part of what patient wishes to communicate with the selection of the expansion icon.

As an illustration, when “Pain” expansion icon 58 on first communication page 38a is selected, as shown in FIG. 3B electronic device 14 can display a second communication page 38b on electronic display 26, which can optionally overlie the first communication page (e.g., the second communication page can be a popup window). Selecting “Pain” expansion icon 58 can also cause electronic device 14 to produce a sound stating that the patient has pain (e.g., “I'm having pain”). The patient can use second communication page 38b's communication icons 46 to communicate pain that the patient is experiencing. For example, a first set of communication icons 46 on second communication page 38b can be for indicating a level of pain that the patient is experiencing (e.g., “No Pain,” “Mild Pain,” “Moderate” pain, “Severe Pain,” or “Worst Pain”) and the patient can select one of those communication icons to cause electronic device 14 to produce a sound conveying (e.g., in one or more words) the patient's level of pain. Furthermore, a second set of communication icons 46 on second communication page 38c can be for indicating a description of pain that the patient is experiencing (e.g., “Sharp,” “Dull,” “Throbbing,” “Burning,” or “Aching”) and the patient can select one of those communication icons to cause electronic device 14 to produce a sound conveying (e.g., in one or more words) a description of the patient's pain. When the patient selects “Choose Pain Location” expansion icon 58 on second communication page 38b, electronic device 14 can display a third communication page 38c that optionally can overlie first communication page 38a (e.g., replacing the second communication page), as shown in FIG. 3C. The patient can use third communication page 38c to indicate where the patient has pain. For example, third communication page 38c can include “Left,” “Right,” “Upper,” and “Lower” communication icons 46 that, when selected, cause electronic device 14 to produce a sound stating that the pain is on the left, right, upper, or lower, respectively, portion of the body. Additionally or alternatively, third communication page 38c can include an avatar 170 and the patient can select a portion of the avatar indicating where the patient is experiencing pain, which can cause electronic device 14 to produce a sound that states what portion of the avatar (e.g., chest, arm, leg, head, back, and/or the like) was selected.

As an additional illustration, when “I Feel” expansion icon 58 on first communication page 38a is selected, as shown in FIG. 3D electronic device 14 can display a fourth communication page 38d on electronic display 26, which can also optionally overlie the first communication page (e.g., like second communication page 38b, the fourth communication page can be a popup window). The patient can use fourth communication page 38d to communicate how the patient is feeling mentally. For example, communication icons 46 on fourth communication page 38d can include “Angry,” “Tired,” “Frustrated,” “Groggy,” “Hopeless,” “Nobody's listening to me,” “Confused,” “Dizzy,” “Sick,” “Worried,” “Cold,” “Hot,” “Excited,” “Happy,” “Thankful,” and/or “Hopeful” communication icons to cause electronic device 14 to produce a sound stating that the patient is angry, tired, frustrated, groggy, hopeless, not being listened to, confused, dizzy, sick, worried, cold, hot, exited, happy, thankful, or hopeful, respectively.

In some embodiments in which there are multiple communication pages 38a-38d, to facilitate the patient's navigation to an appropriate communication page, electronic device 14 can be configured to determine, based at least in part on a sound captured by a microphone 36 of the electronic device, which of the communication pages is a recommended communication page. The sound on which the determination can at least in part be based can be one or more words spoken by another individual such as a caregiver. For example, if the caregiver asks the patient if they are in any pain, electronic device 14 can be configured to determine that second communication page 38b—which, as noted above, can be for indicating a level of pain that the patient is experiencing—is the recommended communication page. Electronic device 14 can automatically display the recommended one of communication pages 38a-38d or display a page that includes an expansion icon 58 that, if selected, causes the electronic device to display the recommended communication page. Such a page with an expansion icon 58 can also include an icon that, if selected, causes electronic device 14 to display the previously-displayed communication page and/or an icon that, if selected, disables the electronic device's determination of a recommended communication page (e.g., if the patient does not wish to use that functionality). Electronic device 14 can use, for example, artificial intelligence predictive pathways to determine which of communication pages 38a-38d is a recommended communication page.

Communication page(s) 38a-38d can have any suitable number of communication and expansion icons 46 and 58 to allow the patient to use electronic device 14 to interact socially with another individual, make requests of the individual, and/or inform the individual about how the patient is feeling, which can facilitate care for the patient. Illustrative communication icons 46 and expansion icons 58 leading to pages with communication icons (e.g., on first communication page 38a) can include, additionally or alternatively to those described above:
a “Social” expansion icon to bring up a communication page with communication icons to produce sounds for social interactions;
a “Breathing/Suction” expansion icon to bring up a communication page with communication icons to produce sounds for delineating breathing issues that the patient is experiencing;
a “Status” expansion icon to bring up a communication page with communication icons to produce sounds requesting the status on pending items;
a “Lie Down” communication icon to produce a sound requesting to recline the bed so that the patient can lie down;
a “Chair” icon that can be a communication icon to produce a sound requesting to be put in a chair or can be an expansion icon to bring up a communication page with communication icons to produce sounds requesting to be put in, adjusted in, and/or removed from a chair;
a “Lights” expansion icon to bring up a communication page with communication icons to produce sounds requesting adjustments to the lights in the room;
a “Mobile” communication icon to produce a sound requesting a mobile device like a phone;
a “Dentures” communication icon to produce a sound requesting dentures;
a “Door” icon that can be a communication icon to produce a sound requesting that a door to the room be closed or an expansion icon to bring up a communication page with communications icons to produce sounds requesting adjustments to the door to the room (e.g., to close or open the door);
an “Adjust Room” expansion icon to bring up a communication page with communication icons to produce sounds for requesting adjustments to the patient's room;
a “Quiet” communication icon to produce a sound requesting silence;
a “Glasses” communication icon to produce a sound requesting glasses;
a “Urinal” icon that can be a communication icon to produce a sound requesting a urinal or an expansion icon to bring up a communication page with communication icons to produce sounds regarding a urinal (e.g., to provide or remove the urinal);
a “TV” icon that can be a communication icon to produce a sound requesting to watch television and/or the television remote or an expansion icon to bring up a communication page with communication icons to produce sounds for making requests regarding the television;
a “Hungry/Thirsty/Ice Chips” expansion icon to bring up a communication page with communication icons to produce sounds for indicating that the patient is hungry, thirsty, or wants ice chips;
a “Sleep” communication icon to produce a sound requesting sleep;
a “Hearing Aid” communication icon to produce a sound requesting a hearing aid;
a “Washcloth” communication icon to produce a sound requesting a washcloth;
a “Call Light” communication icon to produce a sound requesting that the call light be turned on;
a “Position” expansion icon to bring up a communication page with communication icons to produce sounds for requesting a change in the patient's position on the bed;
a “Pillow” icon that can be a communication icon to produce a sound requesting a pillow or an expansion icon to bring up a communication page with communication icons to produce sounds regarding desired adjustments to the patient's pillow;
a “Blanket” icon that can be a communication icon to produce a sound requesting a blanket or an expansion icon to bring up a communication page with communication icons to produce sounds regarding desired adjustments to the patient's blanket;
a “Toothbrush” communication icon to produce a sound requesting a toothbrush;
a “Bath” communication icon to produce a sound requesting a bath;
a “Toilet” icon that can be a communication icon to produce a sound requesting to go to the toilet or an expansion icon to bring up a communication page with communication icons to produce sounds regarding the patient's need to go to the bathroom;
a “People” expansion icon to bring up a communication page with communication icons to produce sounds regarding people the patient wishes to address;
a “Please & Thank you” expansion icon to bring up a communication page with communication icons to produce sounds stating “Please” or “Thank you”; and/or
a “NURSE” icon that can be a communication icon to produce a sound requesting a nurse or an expansion icon to bring up a communication page with communication icons to produce sounds for requesting one of a plurality of options for nurse assistance.

A patient unable to verbally communicate with another individual can use communication icons 46—whether on first communication page 38a or, if there are multiple communication pages, another communication page displayed by selecting one of expansion icon(s) 58—to, via electronic device 14, achieve most if not all of the patient's communicative needs in, for example, a healthcare setting where others are taking care of the patient. For situations where the patient wishes to communicate something not adequately communicable with communication icons 46, one of expansion icon(s) 58 can comprise a keyboard icon that, when selected, causes electronic display 14 to display, on electronic display 26, a keyboard page 62 (e.g., overlying first communication page 38a), as shown in FIG. 3E. Keyboard page 62 can include a plurality of typing icons 66 that can include letters and, optionally, numbers and that a patient can select to type of string of text into a text box 70 of the keyboard page. Typing icons 66 can also include a space icon (e.g., for making a space in text box 70) and a backspace icon for deleting one or more text characters in the text box. Using keyboard page 62, the patient can type the string of text the patient wishes to be communicated into text box 70 and electronic device 14 can be configured to produce a sound, via its speaker(s) 34, dictating the string of text, such as when the patient selects enter icon 74 of the keyboard page after typing the string of text.

Electronic device 14 can be configured to determine, based at least in part on a communication input (e.g., from the patient), which of icons 42 of communication page(s) 38a-38d is a selected icon. The type of communication input that a patient is able to make can depend on, for example, the patient's medical condition and/or what, if any, devices the patient is outfitted with. For example, a patient with one or more hand, arm, and/or shoulder injuries may have limited to no use of at least one of their hands and thus may not be able to use the same to interact with electronic device 14. As another example, a patient that has a neck injury and/or is outfitted with an immobility device like a cervical collar may be unable to move their head such that they may not be able to use head movements to interact with electronic device 14. To accommodate patients that may have different mental and/or sensorimotor limitations, electronic device 14 can have multiple communication modes, wherein for each of the communication modes, the communication input for determining which icon is the selected icon is different when the electronic device is in the communication mode than when the electronic device is in at least one other of the communication modes. Accordingly, a patient that cannot make the communication input of a first communication mode but can make the communication input of a second communication mode may still be able to use electronic device 14 when the electronic device is in the second communication mode.

Electronic device 14's communication modes can include one or more touch-based communication modes, wherein electronic display 26 can comprise a touchscreen and for each of the touch-based communication mode(s), when the electronic device is in the touch-based communication mode, the communication input can comprise an icon selection input that includes touching the touchscreen. When touchscreen 26 is touched at a location disposed over one of icons 42 of communication page(s) 38a-38d, electronic device 14 can be configured to determine that the icon over which the touched location is disposed is the selected icon. For a patient with use of at least one of their hands, the touch-based communication mode(s) can be an easy, intuitive way for the patient to select icons 42 on communication page(s) 38a-38d.

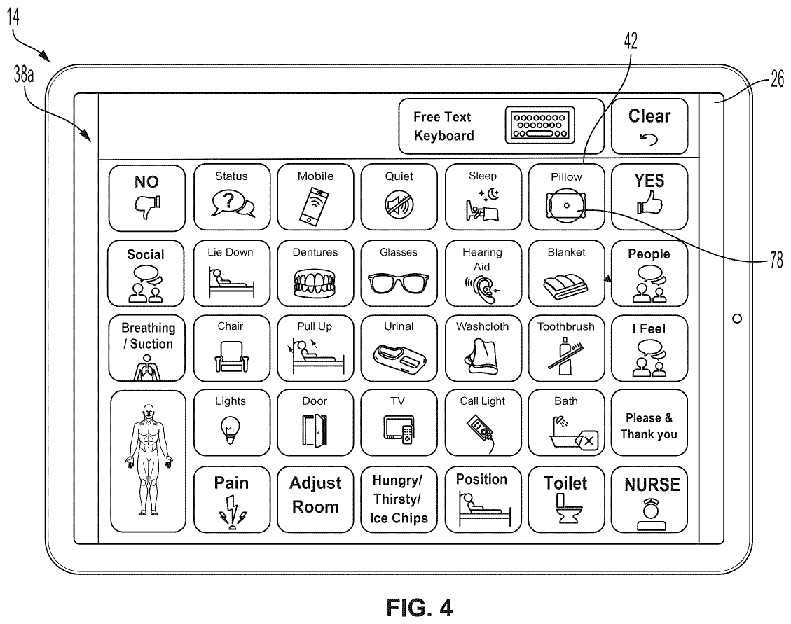

Referring to FIG. 4, the communication modes can also comprise one or more cursor-based communication modes. For each of the cursor-based communication mode(s), when the electronic device is in the cursor-based communication mode, for each of communication page(s) 38a-38d electronic device 14 can be configured to display, on electronic display 26, one or more cursors 78 on the communication page. Cursor(s) 78 can indicate where on the communication page the patient can make a selection using an icon selection input of the communication input. Thus, electronic device 14 can be configured such that when cursor(s) 78 are disposed over one of icons 42 and the patient makes the icon selection input, the electronic device determines that the icon over which the cursor(s) are disposed is the selected icon.

For each of communication page(s) 38a-38d, cursor(s) 78 can move on the communication page without an input from a hand of the patient, rendering the cursor-based communication mode(s) usable for patients that may not be able to tap and touch electronic display 26 of electronic device 14. For at least one of the cursor-based communication mode(s), the communication input from the patient can cause cursor(s) 78 to move. For example, the cursor-based communication mode(s) can include one or more head-based communication mode(s), wherein for each of the head-based communication mode(s), when electronic device 14 is in the head-based communication mode, the communication input can comprise movement of a head of the patient. When electronic device 14 is in one of the head-based communication mode(s), the patient can thus move their head to move cursor(s) 78, e.g., for each of communication page(s) 38a-38d, the electronic device can be configured to determine a position of the cursor(s) on the communication page based at least in part on a position of the head of the patient. Additionally or alternatively, the cursor-based communication mode(s) can include one or more eye-based communication mode(s), wherein for each of the eye-based communication mode(s), when electronic device 14 is in the eye-based communication mode, the communication input can comprise movement of at least one-optionally each—of one or more (e.g., two) eyes of the patient. When electronic device 14 is in one of the eye-based communication mode(s), the patient can thus move their eye(s) to move cursor(s) 78, e.g., for each of communication page(s) 38a-38d, the electronic device can be configured to determine a position of the cursor(s) on the communication page based at least in part on a position of the eye(s) of the patient. Electronic device 14 can determine the position of the head of the patient and/or the position of the eye(s) of the patient using, for example, its camera 30.

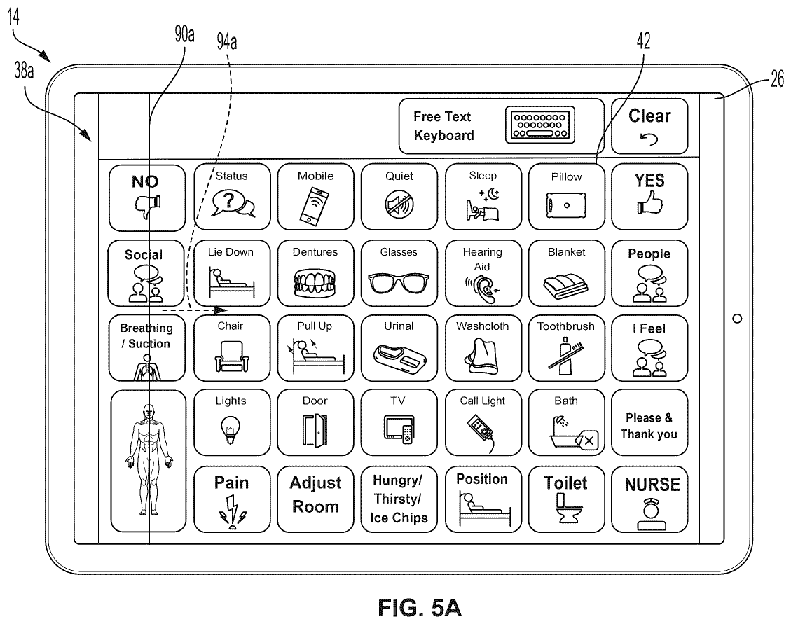

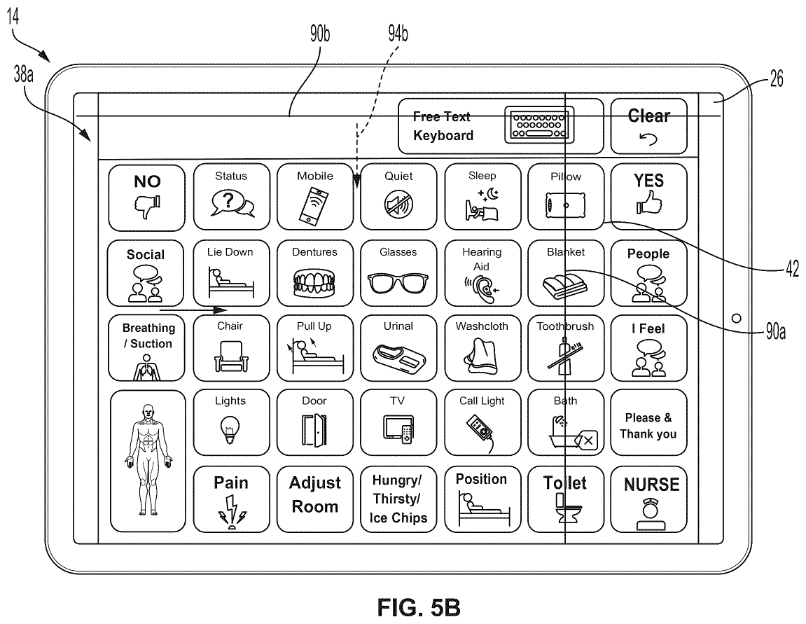

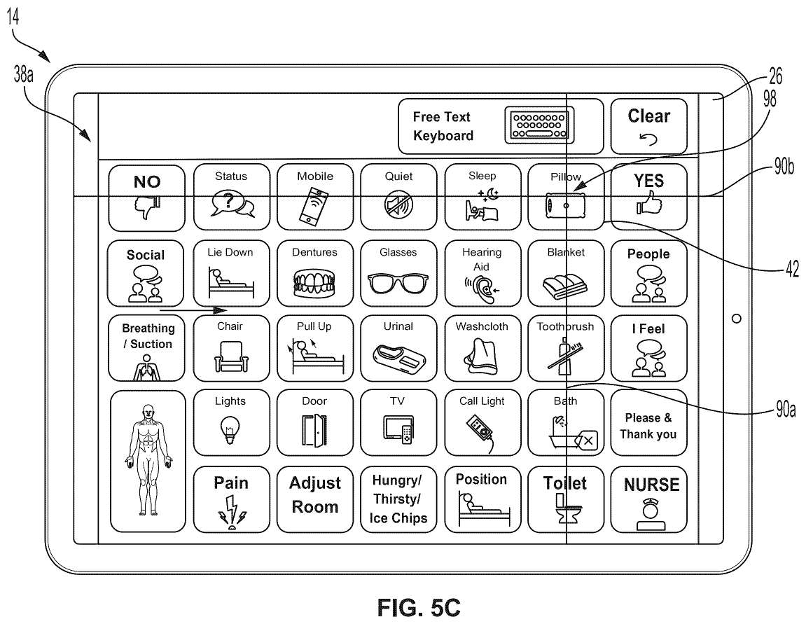

Referring further to FIGS. 5A-5C, for at least one of the cursor-based communication mode(s), electronic device 14 can be configured to move cursor(s) 78 without input from the patient, which can facilitate use by patients who have difficulty making a communication input to move the cursor(s). For example, as shown, the cursor-based communication mode(s) can comprise one or more gliding-cursor-based communication mode(s), wherein for each of the gliding-cursor-based communication mode(s), when electronic device 14 is in the gliding-cursor-based communication mode, cursor(s) 78 can comprise a first linear cursor 90a that extends in a first direction 94a and a second linear cursor 90b that extends in a second direction 94b that is substantially perpendicular to the first direction. The communication input of each of the gliding-cursor-based communication mode(s) can comprise a stop input and, for each of communication page(s) 38a-38d, electronic device 14 can be configured to display, on electronic display 26, first linear cursor 90a moving in second direction 94b (FIG. 5A), wherein when the stop input is made (e.g., by the patient), the electronic device can stop the first linear cursor and display the first linear cursor at a position at which the first linear cursor is stopped (FIG. 5B). After first linear cursor 90a is stopped, electronic device 14 can display second linear cursor 90b moving in first direction 94a (FIG. 5B). When an intersection point 98 at which first linear cursor 90a intersects second linear cursor 90b is disposed over one of icons 42 and the icon selection input is made, electronic device 14 can be configured to determine that the icon over which the intersection point is disposed is the selected icon. In this manner, a patient that cannot tap and touch electronic display 26 and cannot make movements to move cursor(s) 78 may nevertheless be able to select icons 42, as long as the patient can make the stop input and the icon selection input. Additionally, the gliding-cursor-based communication mode(s) may be less demanding on the patient's cognition than the other cursor-based communication mode(s) and thus may be well-suited for patients with cognitive impairments.

The icon selection input and, for the gliding-cursor-based communication mode(s), stop input can comprise one or more actions that even a patient with speech and mobility limitations can make. For example, the icon selection input can include a facial gesture (e.g., captured by electronic device 14's camera 30), a sound (e.g., capture by the electronic device's microphone 36), and/or—for the cursor-based communication mode(s) like the head-based communication mode(s) and eye-based communication mode(s) in which the patient can move cursor(s) 78—maintaining the cursor(s) within an area on one of the communication page(s) for greater than or equal to a threshold dwell time. The facial gesture can be one selected from one or more lower facial gestures (e.g., poking out a tongue, opening a mouth, smiling, and/or grimacing) and/or one or more upper facial gestures (e.g., raising eyebrows, scrunching nose, and/or blinking eye(s)) that the patient can make, and the sound can be selected from one or more sounds that the patient can make with their mouth and/or tongue and that are not speech, such a tongue click, a pop sound, a “P” sound, a “K” sound, a “Ma” sound, and/or a “La” Sound. For each of the gliding-cursor-based communication mode(s), the stop input can include a facial gesture and/or a sound (e.g., any of those described above), and optionally can be the same as the selection input of the gliding-cursor-based communication mode. As described in further detail below, electronic device 14 can be configured to facilitate the selection of the type of icon selection input and stop input that can best allow a particular patient to select one of icons 42.

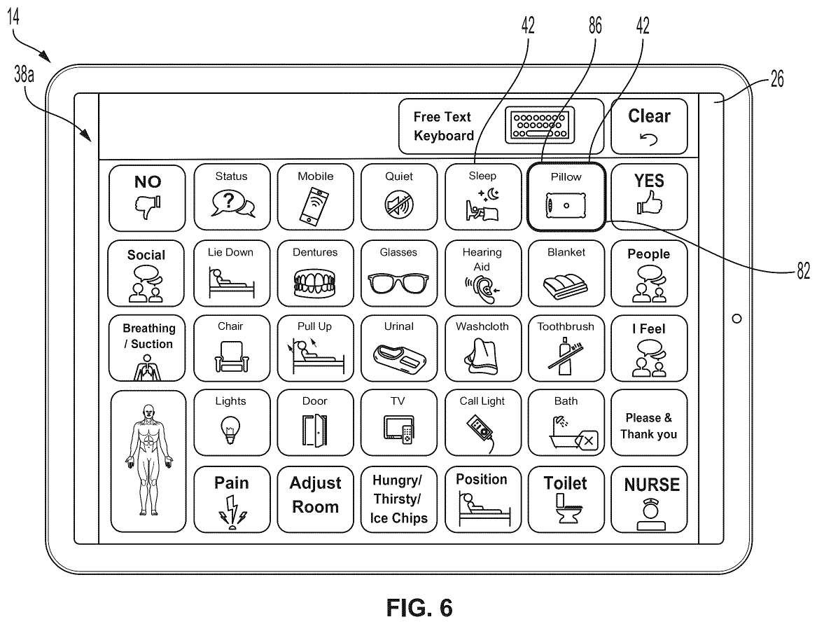

Referring to FIG. 6, the communication mode(s) of electronic device 14 can also include one or more scroll-based communication modes. For each of the scroll-based communication mode(s), when electronic device 14 is in the scroll-based communication mode, the electronic device can be configured to determine which one of icons 42 on communication page(s) 38a-38d is a selectable icon 86 and display, on electronic display 26, an indication 82 regarding which one of the icons is the selectable icon. Indication 82 can comprise, for example, an outline around selectable icon 86 (e.g., as shown), a color of the selectable icon (e.g., that can be different than the color of other ones of icons 42), a cursor on the selectable icon, a size of the selectable icon (e.g., that can be smaller or larger than the size of other ones of the icons), and/or the like. When electronic device 14 is in one of the scroll-based communication mode(s), the communication input can comprise an icon selection input that includes a facial gesture and/or a sound (e.g., any of those described above), wherein for each of icons 42, when the icon is selectable icon 86 and the icon selection input is made, the electronic device can be configured to determine that the icon is the selected icon. The scroll-based communication mode(s) may be well-suited for patients that cannot move cursor(s) 78 in some of the cursor-based communication mode(s).

To allow the patient to selection a desired icon 42, when electronic device 14 is in one of the scroll-based communication mode(s), the electronic device can permit the patient to change or can automatically change which of the icons is selectable icon 86. For example, the scroll-based communication mode(s) can comprise one or more switching-based communication mode(s), wherein for each of the switching-based communication mode(s), when electronic device 14 is in the switching-based communication mode the communication input can comprise a switching input. For each of icons 42, when the icon is selectable icon 86 and the switching input is made, electronic device 14 can be configured to determine that the icon is no longer the selectable icon and that another one of the icons is the selectable icon (e.g., and such that indication 82 indicates that the other icon is now the selectable icon). The patient can thus scroll through icons 42 by making the switching input one or more times until the icon that the patient wishes to select is selectable icon 86, at which point the patient can make the icon selection input to select the icon. Like the icon selection input, he switching input can include a facial gesture and/or a sound (e.g., any of those described above), but can be different than the icon selection input such that electronic device 14 can differentiate between the switching input and the icon selection input.

Additionally or alternatively, the scroll-based communication mode(s) of electronic device 14 can include one or more gliding-switch-based communication modes in which the electronic device can change which of icons 42 is selectable icon 86 without an input from the patient. For each of the gliding-switch-based communication mode(s), when electronic device 14 is in the gliding-switch-based communication mode, for each of icons 42, when the icon has been selectable icon 86 for a threshold selection time, the electronic device can be configured to determine that the icon is no longer the selectable icon and that another one of the icons is the selectable icon. Thus, electronic device 14 can automatically scroll through icons 42 on a communication page for the patient until the patient makes the icon selection input to indicate that the icon that, at the time the icon selection input is made, is selectable icon 86 is the selected icon. While the gliding-switch-based communication mode(s) may require the patient to wait longer for the desired icon 42 to be selectable icon 86 than other switching-based communication mode(s), the gliding-switch-based communication mode(s) may be less demanding on the patient's cognition and thus may be well-suited for patients with cognitive impairments.

In some embodiments, electronic device 14's communication modes can include one or more voice-based communication mode(s) in which icon selection can be based at least in part on speech of the patient. The voice-based communication mode(s) may be useful when, for example, the patient can speak but not in a language that the caregiver can understand.

While for each of the communication modes the communication input can be different than the communication input of at least one other of the communication modes, for at least one of the communication modes, when electronic device 14 is in the communication mode, the electronic device can still be configured to register a selection of one of icons 42 based at least in part on an input that can be used to select one of the icons in another one of the communication modes. That is, as between two communication modes, the communication modes can have different communication inputs if one of icons 42 can be selected with a first type of input when electronic device 14 is in a first one of the communication modes but not when the electronic device is in a second one of the communication modes, even if a second type of input can be used to select one of the icons both when the electronic device is in the first communication mode and when the electronic device is in the second communication mode. As an example, when electronic device 14 is in one of the cursor-based communication mode(s), in one of the scroll-based communication mode(s), and/or in one of the voice-based communication mode(s), the electronic device can still be configured to determine that one of the icons over which a touched location on electronic display 26 is disposed is the selected icon (e.g., as with the touch-based communication mode(s)). This can allow, for example, another user like a caregiver to make selections with touch-based inputs (e.g., to illustrate to the patient how icons 42 on communication page(s) 38a-38d can be used to communicate, to exit the communication page(s), and/or the like), even if the patient must rely on a different input of the communication mode to select one of the icons.

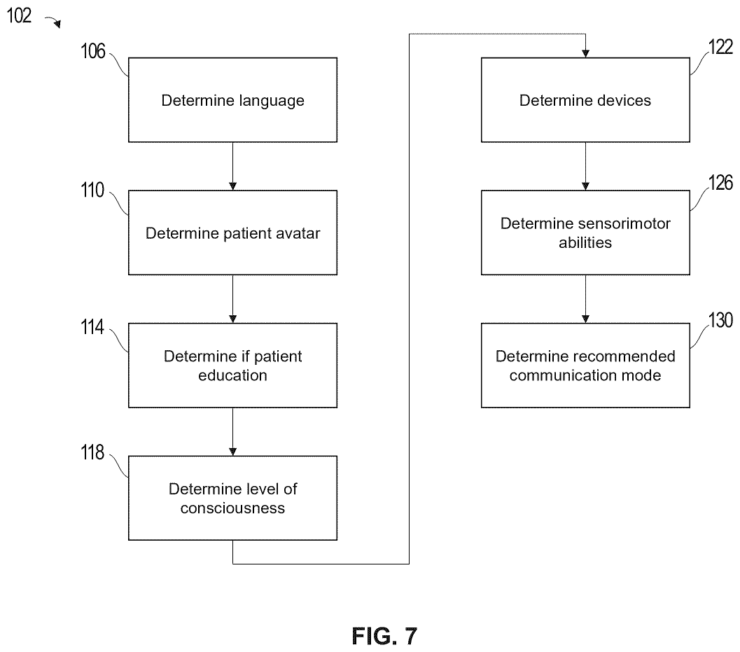

Turning now to FIG. 7, electronic device 14 can be configured to display and receive input for one or more assessments to configure the electronic device for a patient's use, including to determine which of the communication modes is appropriate for the patient. The assessment(s) can be carried out by a caregiver such as a nurse before the patient uses electronic device 14. Shown is an algorithm 102 that electronic device 14 can perform to configure the electronic device.

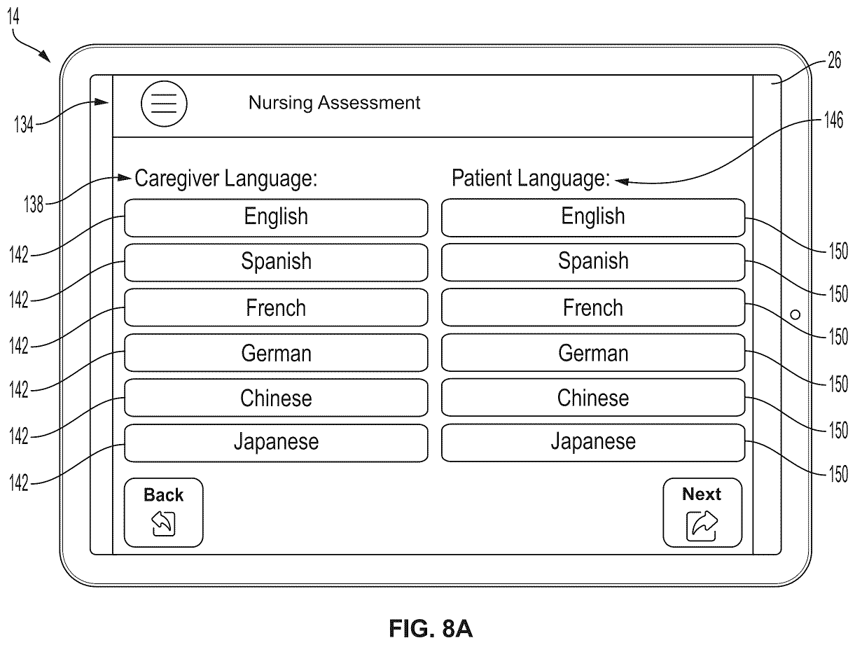

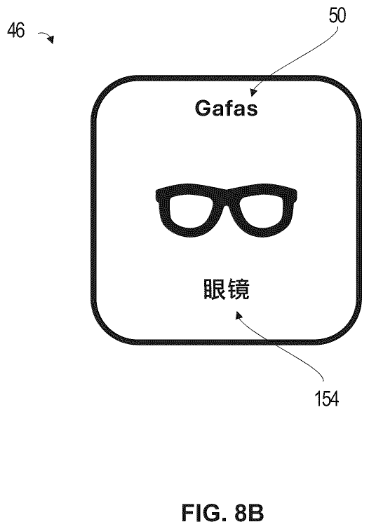

At block 106, electronic device 14 can be configured to determine—and some of the present methods comprise determining-a language that the caregiver is able to understand and/or a language that the patient is able to understand. For example, referring additionally to FIGS. 8A and 8B, electronic device 14 can be configured to display, on electronic display 26, one or more language assessment pages 134 that can include a caregiver language prompt 138 that requests a caregiver language input that comprises a selection of one of a plurality of options 142 regarding a language that the caregiver is able to understand and/or a patient language prompt 146 that requests a patient language input that comprises a selection of one of a plurality of options 150 regarding a language that the patient is able to understand. For example, language assessment pages 134 can display options 146 and 150 as a list of icons (e.g., as shown) or in one or more dropdown menus, and the caregiver can select the appropriate option. The caregiver and patient language inputs can affect how electronic device 14 displays icons 42 and/or the sound that the electronic device is configured to produce when one of communication icons 46 is selected. For example, as shown in FIG. 8B, marking 50 of each icon 42 can be in the language that the patient language input indicates the patient is able to understand, and each icon can also include a marking 154 (e.g., when the patient and caregiver do not understand a common language) that can state the same as marking 50 but is in the language that the caregiver language input indicates the caregiver is able to understand. Furthermore, the sound that electronic device 14 is configured to produce based at least in part on which of communication icons 46 is the selected icon can include a sound in the language that the caregiver language input indicates the caregiver is able to understand such that the caregiver can understand what the patient intends to communicate with the selection of the communication icon.

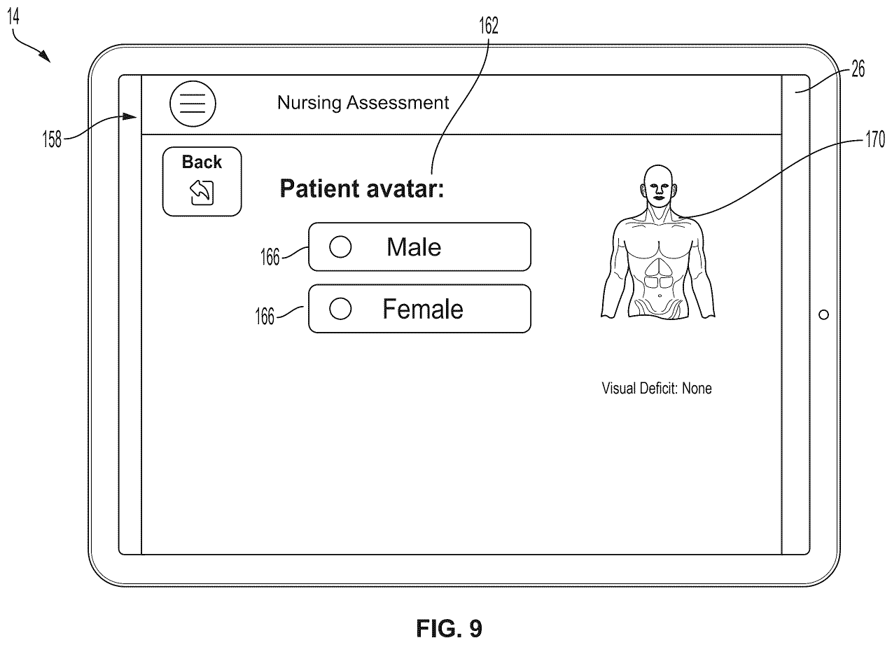

At block 110, electronic device 14 can be configured to determine—and some methods comprise determining—an avatar of the patient. For example, referring additionally to FIG. 9, electronic device 14 can be configured to display, on electronic display 26, one or more avatar assessment pages 158 that can include a patient avatar prompt 162 that requests an avatar input. The avatar input can comprise a selection of one of a plurality of options 166 regarding a gender of the patient. As shown, options 166 can include “Male” and “Female” and can be, for example, listed as icons on avatar assessment page(s) 158 and/or in a dropdown menu thereof, which the caregiver can select based on the gender of the patient. An avatar 170 that can be displayed on avatar assessment page(s) 158 and/or on one or more other of the pages electronic device 14 is configured to display can have the gender that the avatar input indicates is the gender of the patient.

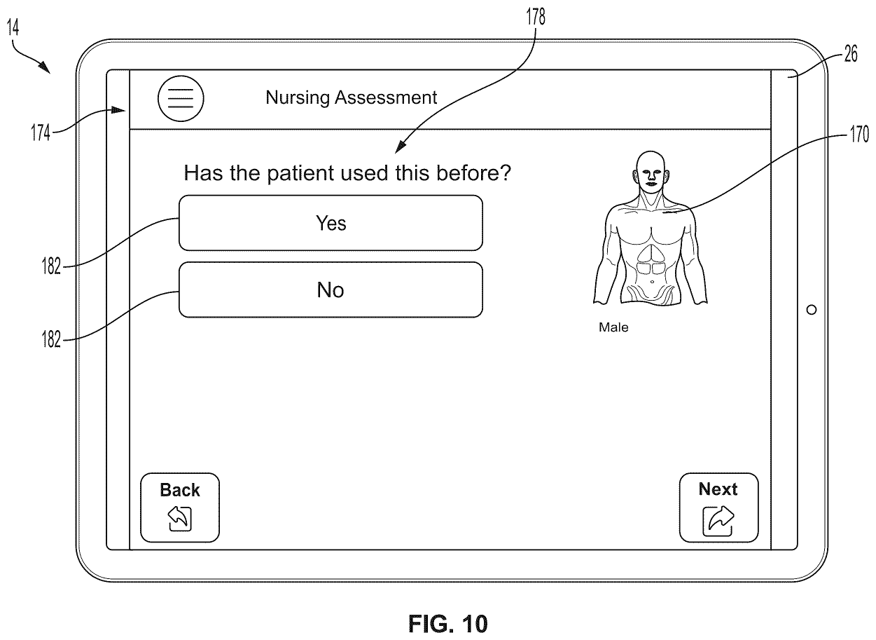

At block 114, electronic device 14 can be configured to determine—and some methods comprise determining—whether to conduct patient education regarding use of the electronic device. For example, referring additionally to FIG. 10, electronic device 14 can be configured to display, on electronic display 26, one or more experience assessment pages 174 that can include a patient experience prompt 178 that requests an experience input. The experience input can comprise a selection of one of a plurality of options 182 regarding whether the patient has used electronic device 14 before to select communication icons 46 to communicate, the options including “Yes” and “No.” If the experience input indicates that the patient has used electronic device 14 before to select communication icons 46 to communicate, the electronic device can determine that patient education is not needed. If the experience input indicates that the patient has not used electronic device 14 before, the electronic device can determine that the patient should be educated and can, either when the experience input is entered or thereafter, display a patient education page on which the patient can learn how to use communication page(s) 38a-38d.

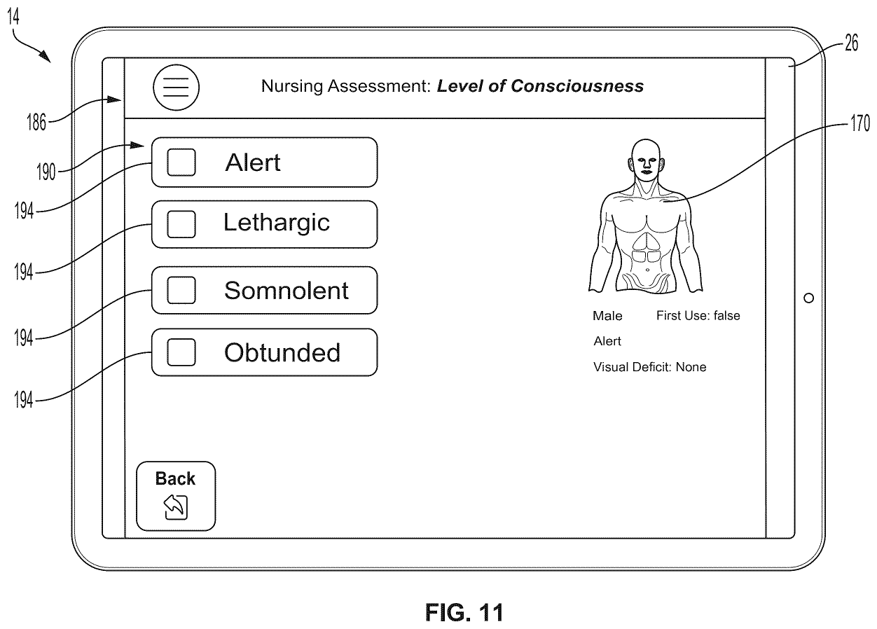

At block 118, electronic device 14 can be configured to determine—and some methods comprise determining—a level of consciousness of the patient. For example, referring additionally to FIG. 11, electronic device 14 can be configured to display, on electronic display 26, one or more consciousness assessment pages 186 that include a prompt 190 that requests a consciousness input. The consciousness input can comprise a selection of one of a plurality options 194 regarding a level of consciousness of the patient. Such options 194 can include at least a first level of consciousness (e.g., alert) and a second level of consciousness that is lower than the first level of consciousness, such as alert, lethargic, somnolent, and/or obtunded. Consciousness assessment page(s) 186 can include, for example, a list of icons for options 194 or a dropdown menu including the options such that the caregiver can select one of them. Which of options 194 regarding the level of consciousness of the patient is selected can affect whether electronic device 14 continues to further assessment page(s) and/or communication page(s) 38a-38d. For example, if the consciousness input indicates that the patient is obtunded or, optionally, somnolent, electronic device 14 may not proceed to further assessment page(s) and/or communication page(s) 38a-38d (e.g., because the patient may not have an adequate level of consciousness to reliably use the communication page(s)). Additionally or alternatively, the consciousness input can be used in the determination of which of the communication modes is recommended for the patient as described in further detail below.

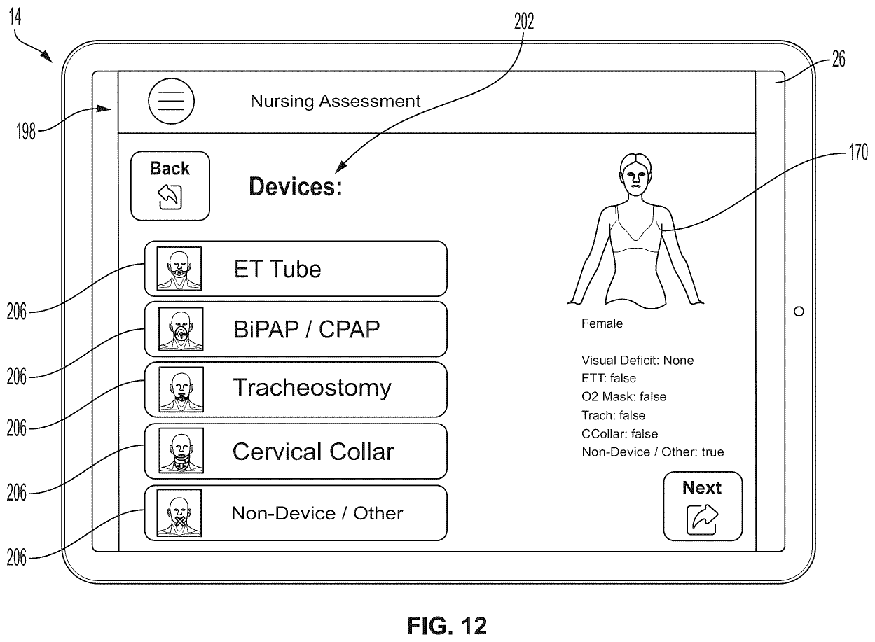

At block 122, electronic device 14 can be configured to determine—and some methods comprise determining—whether the patient is outfitted with one or more devices. For example, referring additionally to FIG. 12, electronic device 14 can be configured to display, on electronic display 26, one or more device assessment pages 198 that can include a device prompt 202 that requests a device input comprising a selection of at least one of a plurality of options 206 regarding whether the patient is outfitted with one or more devices. The device(s) of options 206 can include one or more, optionally a plurality of, medical devices relevant to the patient's sensorimotor abilities and thus to which of the communication inputs the patient can make. For example, the device(s) can include one or more immobility devices configured to inhibit movement of the head of the patient such as a cervical collar, one or more mouth devices configured to be disposed on and/or through the mouth of the patient such as an endotracheal (ET) tube and/or a bilevel positive airway pressure (BiPap) and/or continuous positive airway pressure (CPAP) ventilator, and/or a tracheostomy tube. Options 206 can also include an option indicating that the patient is not outfitted with a device and/or is outfitted with a device other than the device(s) of the other of the options. Device assessment page(s) 198 can display options 206 as a list of icons (e.g., as shown) or in a dropdown menu, allowing the caregiver to select one or more of the options. Furthermore, device assessment page(s) 198 can include avatar 170 and, for each of options 206 that is selected, electronic device 14 can display the device, if any, of the selected option on the avatar to facilitate the caregiver's ability to keep track of which of the options is selected. As explained in further detail below, electronic device 14 can use the device input to determine one or more sensorimotor abilities of the patient and/or to determine which of a plurality of options regarding the patient's sensorimotor abilities should be preselected in the subsequent sensorimotor assessment.

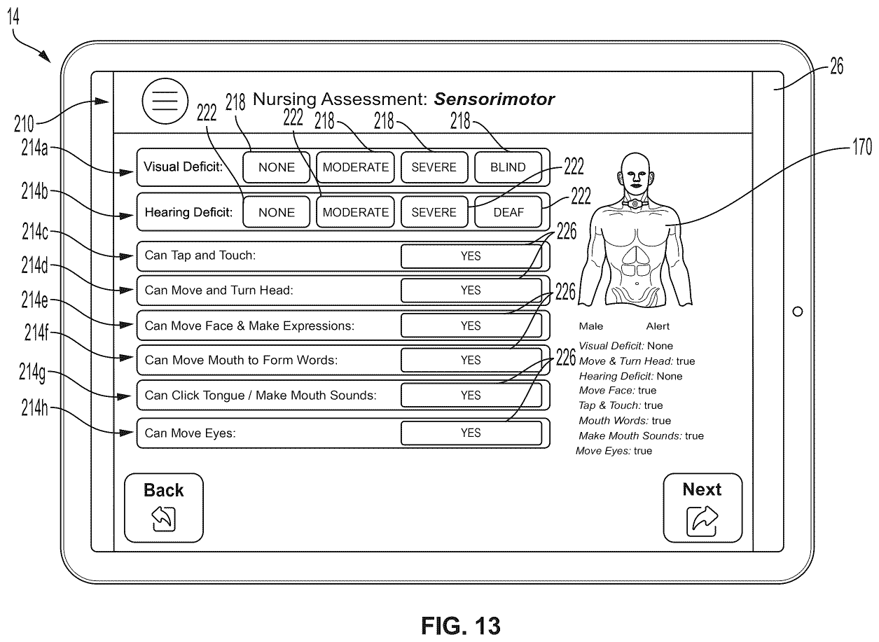

At block 126, electronic device 14 can be configured to determine—and some methods comprise determining—a plurality of sensorimotor abilities of the patient. For example, referring additionally to FIG. 13, electronic device 14 can be configured to display, on electronic display 26, one or more sensorimotor assessment pages 210 that include a plurality of prompts 214a-214h that each request a sensorimotor input. For each of prompts 214a-214h, the sensorimotor input that the prompt requests can comprise a selection of one of a plurality of options regarding a sensorimotor ability of the patient. Prompts 214a-214h can comprise one or more prompts to assess the patient's vision and/or hearing such that electronic device 14 can accommodate a deficit therein. For example, prompts 214a-214h can comprise a visual-acuity prompt 214a, wherein the sensorimotor input that the visual-acuity prompt requests comprises a selection of one of a plurality of options 218 regarding a visual acuity of the patient that can include at least a first level of visual acuity and a second level of acuity that is lower than the first level of visual acuity; as shown, those options can include no visual deficit, a moderate visual deficit, a severe visual deficit, and blind. As explained in further detail below, when the input indicates that the patient has a visual deficit above a threshold deficit (e.g., the second level of visual acuity, such as a severe visual deficit), electronic device 14 can utilize larger selectable options to accommodate the visual deficit. Additionally or alternatively, prompts 214a-214h can comprise a hearing prompt 214b, wherein the sensorimotor input that the hearing prompt requests comprises a selection of one of a plurality of options 222 regarding a hearing ability of the patient that include at least a first level of hearing and a second level of hearing that is lower than the first level of hearing; as shown, those options can include no hearing deficit, a moderate hearing deficit, a severe hearing deficit, and deaf. For one or more of the hearing levels (e.g., moderate hearing deficit and/or severe hearing deficit), when the hearing level is selected, electronic device 14 can be configured to increase the volume at which its speaker(s) 34 play one or more sounds (e.g., for the patient), relative to the volume when the selection indicates the patient has no hearing deficit. Additionally or alternatively, for one or more of the hearing levels (e.g., moderate hearing deficit, severe hearing deficit, and/or deaf), when the hearing level is selected, electronic device 14 can be configured to increase visual feedback for icon selections (e.g., by increasing the brightness of indication 82 and/or of an indication that occurs when one of icons 42 is selected), display, on electronic display 26, that transcription of speech to text on the electronic display is recommended, and/or automatically enable such transcription.

Prompts 214a-214h of sensorimotor assessment page(s) 210 can also include one or more prompts to assess one or more actions that the patient can take to select icons 42. For example, prompts 214a-214h can include:
a touch prompt 214c, wherein the sensorimotor input that the touch prompt requests comprises a selection of one of a plurality of options 226 (e.g., yes or no) regarding whether the patient can touch electronic display 26 (e.g., the touchscreen) with one of a plurality of fingers of the patient;
a head-movement prompt 214d, wherein the sensorimotor input that the head-movement prompt requests comprises a selection of one of a plurality of options (e.g., yes or no) regarding whether the patient can move their head (e.g., vertically and horizontally);
a facial-gesture prompt 214e, wherein the sensorimotor input that the facial-gesture prompt requests comprises a selection of one of a plurality of options (e.g., yes or no) regarding whether the patient can make a facial gesture (e.g., the ability to move the face and make facial expressions);
a mouth-word prompt 214f, wherein the sensorimotor input that the mouth-word prompt requests comprises a selection of one of a plurality of options (e.g., yes or no) regarding whether the patient can move their mouth to form words;
a sound prompt 214g, wherein the sensorimotor input that the sound prompt requests comprises a selection of one of a plurality of options (e.g., yes or no) regarding whether the patient can make a sound with their mouth and/or tongue; and/or
an eye-movement prompt 214h, wherein the sensorimotor input that the eye-movement prompt requests comprises a selection of one of a plurality of options (e.g., yes or no) regarding whether the patient can move their eye(s).

Sensorimotor assessment page(s) 210 can display options 218, 222, and 226 as, for example, icons including the options, in a dropdown menu, and/or in an icon that can be toggled between the options. In the embodiment shown, each of options 218 for visual-acuity prompt 214a and each of options 222 for hearing prompt 214b can be displayed in a respective icon, while for each of sensorimotor prompts 214d-214h for which options 226 comprise “yes” or “no,” a single icon can be toggled between “yes” and “no” when selected.

The devices that the patient is outfitted with can affect the patient's sensorimotor abilities. For example, as noted above, an immobility device like a cervical collar can inhibit movement of the patient's head, and a mouth device configured to be disposed on and/or through the patient's mouth can prevent the patient from moving their mouth to form words, making a sound with their mouth and/or tongue, and/or making a facial gesture. As such, the device input requested by device prompt 202 can be relevant to one or more of the sensorimotor inputs for sensorimotor prompts 214a-214h. To facilitate the efficient and complete sensorimotor assessment, electronic device 14 can be configured to preselect, for at least one of prompts 214a 214d, one of options 218, 222, or 226 for the prompts based the device input requested by device prompt 202. For example, when the device input indicates that the patient is outfitted with at least one of the immobility device(s) like a cervical collar, electronic device 14 can be configured to preselect one of options 226 for head-movement prompt 214d indicating that the patient cannot move their head (e.g., “no”). As another example, when the device input indicates that the patient is outfitted with at least one of the mouth device(s) such as an ET tube and/or a BiPap/CPAP ventilator, electronic device 14 can be configured to preselect one of options 226 for facial-gesture prompt 214e indicating that the patient cannot make a facial gesture (e.g., “no”) (e.g., when outfitted with an ET tube), one of the options for mouth-word prompt 214g indicating that the patient cannot move their mouth to form words (e.g., “no”) (e.g., when outfitted with an ET tube and/or a BiPAP/CPAP ventilator), and/or one of the options for sound prompt 214g indicating that the patient cannot make a sound with their mouth and/or tongue (e.g., “no”) (e.g., when outfitted with an ET tube and/or a BiPAP/CPAP ventilator). Sensorimotor assessment page(s) 210 can include an indication regarding which of options 218, 222, and/or 226 is preselected to notify the user of the same. The user can, in addition to selecting one of the options for each of sensorimotor prompt(s) 214a-214h for which no preselection is made, override the preselected option for each of the sensorimotor prompt(s) for which a preselection is made, if appropriate, by selecting a different one of the options for the sensorimotor prompt.

At block 130, electronic device 14 can be configured to determine—and some methods comprise determining—which of the communication modes of the electronic device is a recommended communication mode. The recommended communication mode can be one that accommodates the patient's sensorimotor abilities to efficiently interact with electronic device 14 to select icons 42 while also allowing the electronic device to reliably detect inputs made by the patient. Electronic device 14 can thus be configured to determine which of the communication modes in the recommended communication mode based at least in part on the sensorimotor inputs and, optionally, one or more inputs from other assessment(s) such as the consciousness input and/or device input. Utilizing such inputs, electronic device 14 can determine the recommended communication mode in a way that prioritizes communication modes that the patient can use and that involve inputs that the electronic device can more-reliably detect, allowing the electronic device to more consistently be configured in a way that better facilitates patient communication, compared to, for example, a caregiver's unassisted selection of the communication mode. Electronic device 14 can automatically enter into the recommended communication mode and/or display, on electronic display 26, which of the communication modes is the recommended communication mode to inform the user (e.g., caregiver) of which communication mode is recommended and thus should be selected.

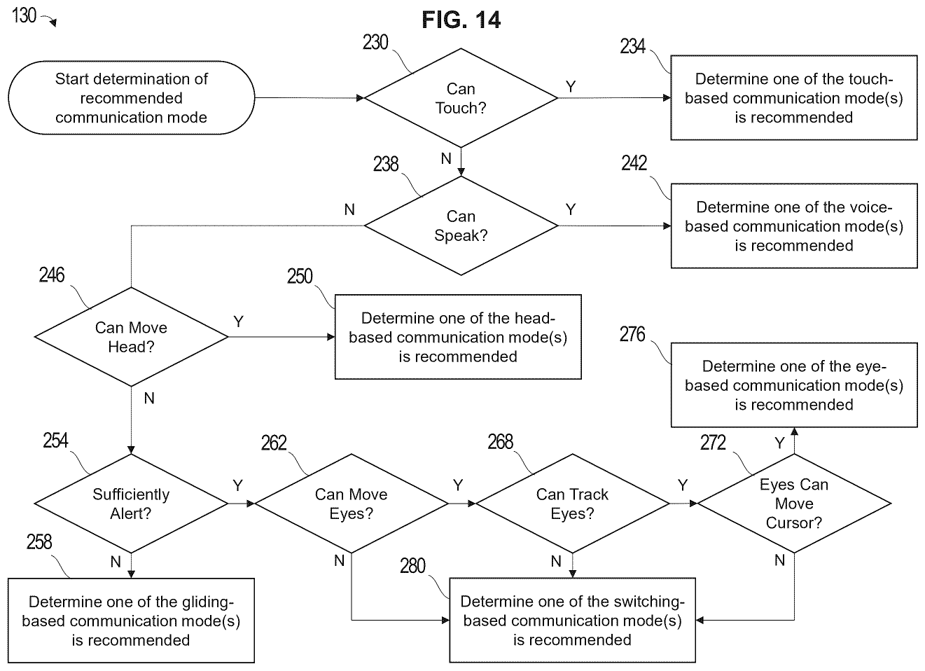

Turning to FIG. 14, shown is an algorithm for how electronic device 14 can determine which of the communication modes is the recommended communication mode. When the communication modes include one or more touch-based communication modes, at block 230 electronic device 14 can be configured to determine—and some methods comprise determining-whether the patient can touch electronic display 26 (e.g., the touchscreen) with one of their fingers. That determination can be based on, for example, the sensorimotor input requested by touch prompt 214c. The touch-based communication modes can be the most intuitive for the patient to use and the communication input thereof can be one that electronic device 14 can most-reliably detect, compared to other communication modes. Thus, when the sensorimotor input that touch prompt 214c requests indicates that the patient can touch the touchscreen with one of their fingers, at block 234 electronic device 14 can be configured to determine—and some methods comprise determining—that one of the touch-based communication mode(s) is the recommended communication mode.

If the sensorimotor input for touch prompt 214c indicates that the patient cannot touch the touchscreen with one of their fingers and electronic device 14 has voice-based communication mode(s), at block 238 the electronic device can be configured to determine—and some methods comprise determining—whether the patient can speak. If, based at least in part on, for example, the device input for device prompt 202 and/or the sensorimotor input for mouth-word prompt 214f, electronic device 14 determines that the patient can speak, at block 242 the electronic device can be configured to determine—and some methods comprise determining—that one of the voice-based communication mode(s) is recommended.

If electronic device 14 determines that the patient cannot touch electronic display 26 with one of their finger(s) (and, optionally, that the patient cannot speak), it can proceed to determine that another one of the communication modes (e.g., one of the cursor-based communication mode(s) or one of the scroll-based communication mode(s)) is the recommended communication mode. Such communication modes, while less efficient and intuitive for selecting icons 42, can better accommodate the patient's sensorimotor impediments. Of the cursor-based and scroll-based communication modes, subject to the patient's abilities, electronic device 14 can give priority to the cursor-based communication mode(s) in which the patient actively moves cursor(s) 78, such as the head-based communication mode(s) and the eye-based communication mode(s), because those communication modes can allow the patient to more-quickly select one of icons 42 compared to the scroll-based communication mode(s) and gliding-cursor-based communication mode(s). And electronic device 14 can further prioritize the head-based communication mode(s) over the eye-based based communication mode(s), as the head-based communication mode(s) can be less demanding on the patient's cognition and the electronic device may be able to more reliably detect the head movements used to move cursor(s) 78 in the head-based communication mode(s) than the eye movements used to move the cursor(s) in the eye-based communication mode(s).

To effect such a prioritization, at block 246 electronic device 14 can be configured to determine—and some methods comprise determining—whether the patient can move their head, such as based on what is indicated by the sensorimotor input that head-movement prompt 214d requests. To prioritize the head-based communication mode(s), electronic device 14 can be configured—and some methods can be performed-such that, if the sensorimotor input indicates that the patient can move their head, none of the scroll-based communication mode(s), eye-based communication mode(s), or gliding-cursor-based communication mode(s) are determined to be the recommended communication mode. And when electronic device 14 has determined (e.g., at block 230 and block 238) that the patient who can move their head cannot touch electronic display 26 with one of their fingers (and, optionally cannot speak), at block 250 the electronic device can determine—and some methods comprise determining—that one of the head-based communication mode(s) is the recommended communication mode.

If the sensorimotor input indicates that the patient cannot move their head, electronic device 14 can proceed to determine that another one of the communication modes is the recommended communication mode. To do so, at block 254 electronic device 14 can be configured to determine—and some methods comprise determining—whether the patient has an alertness above a threshold alertness. While the eye-based communication mode(s) and switching-based communication mode(s) may allow faster selection of one of icons 42 than the gliding-based communication mode(s), they be more cognitively demanding and thus may not be well-suited for a patient whose alertness is below the threshold alertness. Thus, if it is determined that the patient's alertness is below the threshold alertness—such as if the consciousness input indicates that the patient has the second level of consciousness (e.g., lethargic)—at block 258 electronic device 14 can be configured to determine—and some methods comprise determining—that one of the gliding-based communication mode(s) (e.g., one of the gliding-cursor-based communication mode(s) or one of the gliding-switch-based communication mode(s)) is the recommended communication mode.

If electronic device 14 determines that the patient's alertness is above the threshold alertness—such as if the consciousness input indicates that the patient has the first level of consciousness (e.g., alert)—the electronic device can be configured to make one or more determinations to determine whether one of the eye-based communication mode(s) or one of the switching-based communication mode(s) is the recommended communication mode. As shown, at blocks 262, 268, and 272 electronic device 14 can be configured to determine—and some methods comprise determining—whether the patient can move their eye(s), whether the electronic device can track the patient's eye(s), and whether the patient can move cursor(s) 78 with their eye(s), respectively. For example, the sensorimotor input that eye-movement prompt 214h requests can indicate whether the patient can move their eye(s), if the patient can move their eye(s) electronic device 14 can determine whether its camera 30 can visualize the patient's eye(s) for tracking, and if the patient's eye(s) can be tracked the electronic device can display an assessment to determine if the patient can move cursor(s) 78 with their eye(s) (e.g., where the patient can be educated and oriented with the eye-based movements of the cursor(s) and the electronic device can be calibrated to the patient's eye movements). If the determination at each of blocks 262, 268, and 272 is yes, then a sufficiently-alert patient may be able to use electronic device 14 when it is in one of the eye-based communication mode(s), and because the eye-based communication mode(s) may allow faster selections of icons 42 than the switching-based communication mode(s), electronic device 14 can be configured—and some methods can be performed-such that none of the switching-based communication mode(s) are determined to be the recommended communication mode when those determinations are made. Thus, when electronic device 14 determines that the patient cannot touch, speak, or move their head but is sufficiently alert and satisfies the determinations at blocks 262, 268, and 272, at block 276 the electronic device can be configured to determine—and some methods comprise determining—that one of the eye-based communication mode(s) is recommended. When electronic device 14 instead determines at block 262 that the patient cannot move their eye(s), at block 268 that it cannot track the patient(s) eyes, or at block 272 that the patient cannot move cursor(s) 78 with their eye(s), at block 280 the electronic device can be configured to determine—and some methods comprise determining—that one of the switching-based communication mode(s) is the recommended communication mode.

Electronic device 14 can also utilize inputs from at least one of the assessments (e.g., the sensorimotor and/or device inputs) to, when determining which of the communication modes is recommended, further accommodate patient impediments beyond those described above and/or determine what type of icon selection input, switching input, and/or stop input to employ.

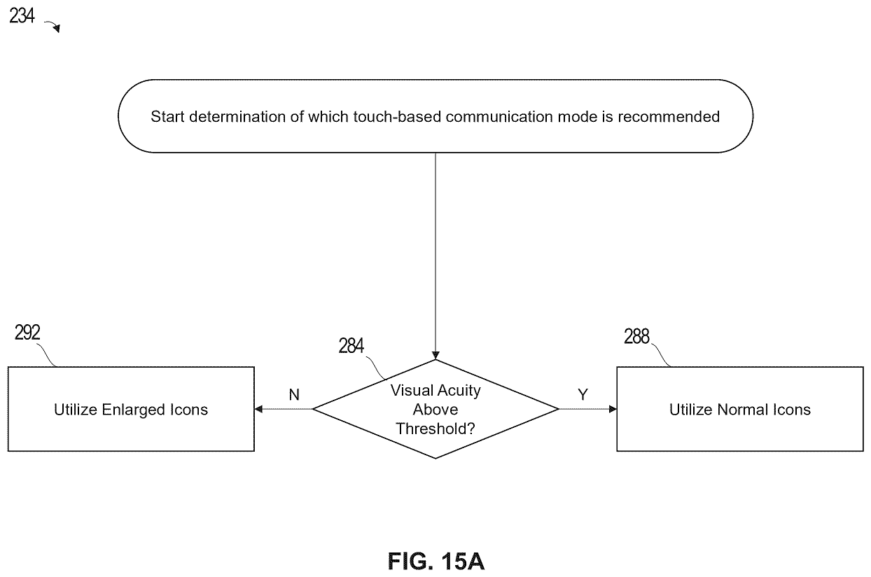

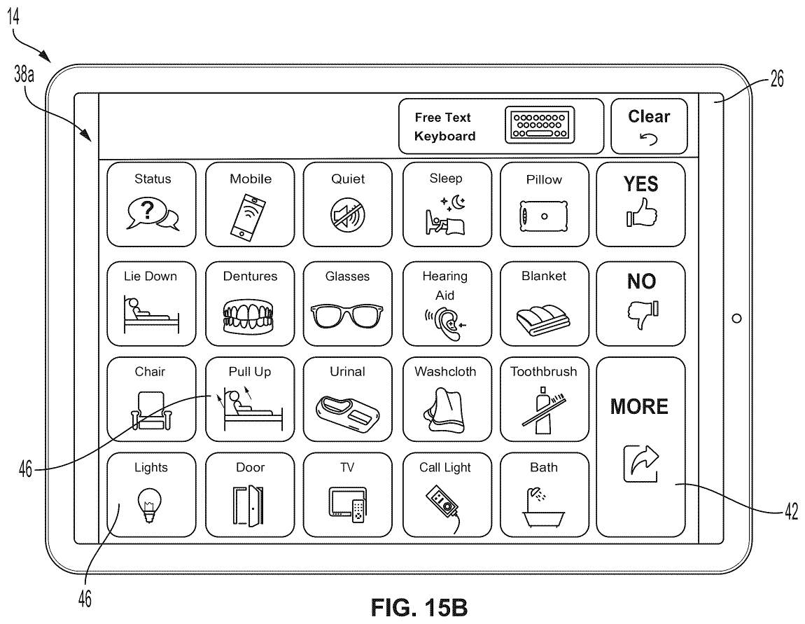

For example, as noted above electronic device 14 can be configured to accommodate visually-impaired patients by enlarging at least some of icons 42. For each of the communication modes of a first portion of the communication modes, when electronic device 14 is in the communication mode, each of communication icons 46 can have a first area, and for each of the communication modes of a second portion of the communication modes, when the electronic device is in the communication mode, each of the communication icons can have a second area that is larger than the first area (e.g., for visually-impaired patients). The option for normal or enlarged communication icons 46 can be available for at least one—up to and including each—of the subset of communication modes (e.g., for the touch-based communication mode(s), voice-based communication mode(s), head-based communication mode(s), eye-based communication mode(s), switching-based communication mode(s), and/or gliding-based communication mode(s)). That is, at least one-up to and including each—of the subset of communication modes can include at least one communication mode of the first portion of communication modes and at least one communication mode of the second portion of communication modes. As an illustration, and referring to FIGS. 15A and 15B, when determining which touch-based communication mode is the recommended communication mode at block 234, at block 284 electronic device 14 can be configured to determine—and some methods comprise determining—whether a visual acuity of the patient is above a threshold level of visual acuity. If yes (e.g., if the sensorimotor input that visual-acuity prompt 214a requests indicates that the patient has the first level of visual acuity), at block 288 electronic device 14 can be configured to determine—and some methods comprise determining—that the touch-based communication mode with normal-sized icons (e.g., the touch-based communication mode that is part of the first portion of communication modes) is the recommended communication mode. If not (e.g., if the sensorimotor input that visual-acuity prompt 214a requests indicates that the patient has the second level of visual acuity), at block 292 electronic device 14 can be configured to determine—and some methods comprise determining—that the touch-based communication mode with enlarged icons (e.g., the touch-based communication mode that is part of the second portion of communication modes) is the recommended communication mode. The same determination can be done for the other subsets of communication modes.

FIG. 15B illustrates one of communication page(s) 38a-38d when electronic device 14 is in one of the communication modes with enlarged communication icons 46 (e.g., that is part of the second portion of communication modes). As shown, each of communication icons 46 has a larger area than when electronic device 14 is in one of the communication modes of the first portion of communication modes (FIG. 3A). Because communication icons 46 are larger, fewer may fit on a single one of communication page(s) 38a-38d. Thus, when electronic device 14 is in one of the communication modes that is part of the second portion of communication modes, there can be (1) more communication pages 38a-38d to accommodate the larger communication icons 46 that might not otherwise fit on the communication pages and (2) at least one additional expansion icon 58 for accessing the additional communication page(s).

As noted above, electronic device 14 can also be configured to determine, for each of the cursor-based communication mode(s) and/or scroll-based communication mode(s), which one of two or more types of icon selection inputs is the icon selection input, the types including one or more facial gestures, one or more sounds, and/or—for the head-based and eye-based communication modes—a dwell time input in which icon(s) 78 are maintained within an area on one of communication page(s) 38a-38d for greater than or equal to a threshold dwell time. The patient may be able to more-reliably select icons 42 with some of the types of icon selection inputs than with others, and some types may be easier and/or more intuitive for the patient. For example, electronic device 14 may be able to more-reliably detect the facial gesture(s) than the sound(s), as ambient noises—especially in the healthcare setting—can disrupt the sound(s). And of the facial gesture(s), it may be easier and more reliable for the patient to select one of icons 42 by making a lower facial gesture like poking out the tongue, opening the mouth, smiling, and/or grimacing than by making an upper facial gesture such as raising eyebrows, scrunching nose, and/or blinking eye(s). This is because, for example, such upper facial gestures may be more difficult to detect and/or may be actions that the patient often performs involuntarily. Electronic device 14 can thus, subject to the patient's abilities, prioritize the facial gesture(s)—and especially the lower facial gesture(s)—over the sound(s) as the icon selection input when determining the recommended communication mode.

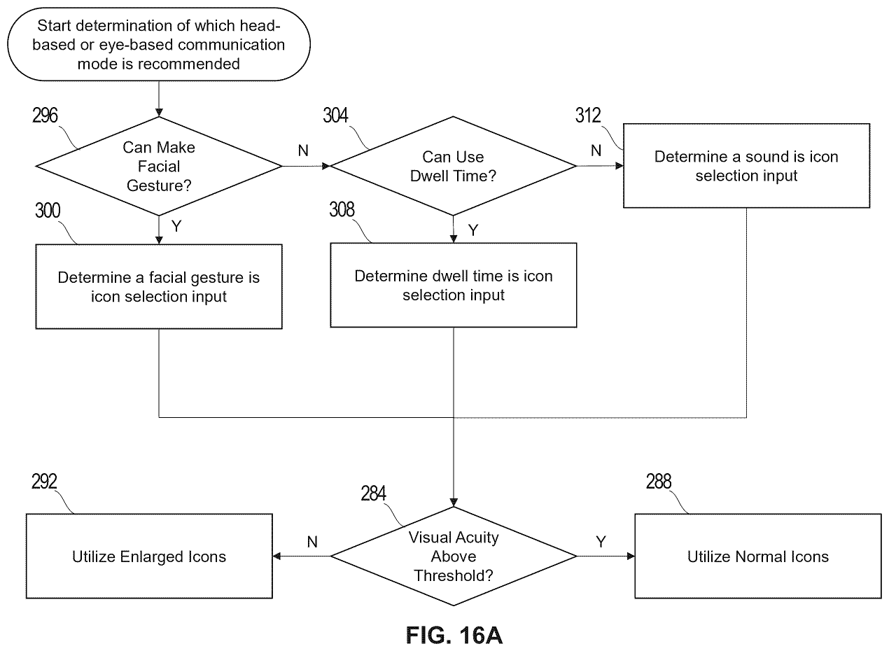

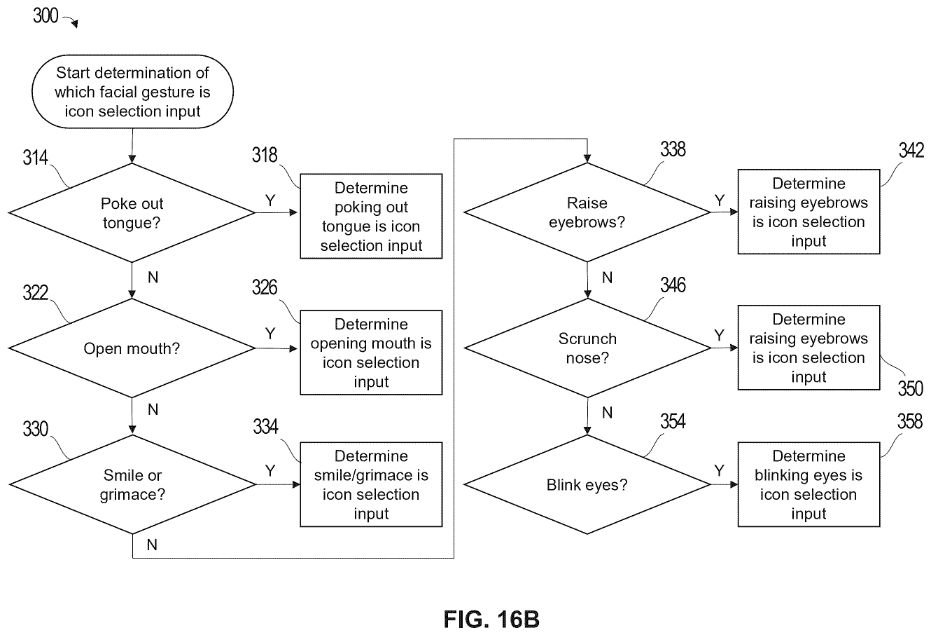

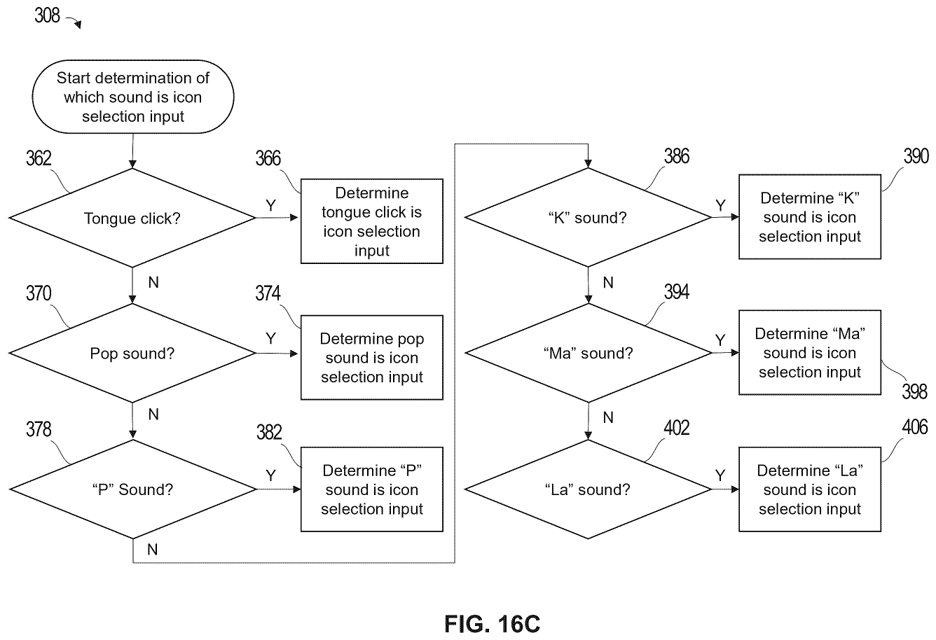

Referring to FIGS. 16A-16C, illustrated is how electronic device 14 can achieve such prioritization when, at block 250 or block 276 (FIG. 14), it determines that one of the head-based or eye-based, respectively, communication modes is the recommended communication mode. As shown in FIG. 16A, at block 296 electronic device 14 can be configured to determine—and some methods comprise determining—whether the patient can make a facial gesture. This determination can be based at least in part on what the sensorimotor input requested by facial-gesture prompt 214g indicates. If the sensorimotor input requested by facial-gesture prompt 214g indicates that the patient can make a facial gesture, then at block 300 electronic device 14 can be configured to determine—and some methods comprise determining—that a facial gesture is the icon selection input.

As shown in FIG. 16B, when there are multiple facial gestures to select from like one or more lower facial gestures and/or one or more upper facial gestures, electronic device 14 can determine which of the facial gestures is the icon selection input. When doing so, electronic device 14 can select the facial gesture that the patient can most-reliably use to select one of icons 42. For example, if the types of facial gestures include lower facial gesture(s) and upper facial gesture(s), when electronic device 14 determines that the patient is capable of making at least one of the lower facial gesture(s), it can be configured—and some methods can be performed—such that none of the upper facial gesture(s) are determined to be the icon selection input. When prioritized in this manner, the facial gestures can include, from highest priority to lowest priority, poking out the tongue, opening the mouth, smiling or grimacing, raising eyebrows, scrunching nose, and/or blinking eye(s). To determine which of such facial gestures is the icon selection input, at block 314 electronic device 14 can be configured to first determine—and some methods comprise determining—whether the patient can poke out their tongue. That being the most-preferred facial gesture, if the determination at block 314 is yes, then at block 318 electronic device 14 can be configured to determine—and some methods comprise determining—that poking out the tongue is the icon selection input. If the determination is no, at blocks 322, 330, 338, 346, and 354, electronic device 14 can be configured to successively determine—and some methods comprise determining—whether the patient can open their mouth, smile or grimace, raise their eyebrows, scrunch their nose, or blink their eye(s), respectively, until one of the determinations is yes, in which case the first of those facial gestures that the patient can make can, at one of blocks 326, 334, 342, 350, or 358, respectively, be determined to be the icon selection input. At least one of these determinations can be based at least in part on the device input. For example, when the device input indicates that the patient is outfitted with at least one of the mouth device(s) that can inhibit mouth movements, electronic device 14 can be configured—and some methods can be performed—such that none of the lower facial gesture(s) (e.g., poking out the tongue, opening the mouth, smiling, and/or grimacing) is the icon selection input. Furthermore, at least one of these determinations, such as whether the patient can raise their eyebrows, scrunch their nose, or blink their eye(s), can be based at least in part on an input from a user (e.g., as requested by a prompt of the sensorimotor assessment page(s)).

If at block 296 electronic device 14 determines that the patient cannot make a facial gesture, the electronic device can be configured to determine that another one of the types of selection inputs is the icon selection input. Using dwell time as an icon selection input may be more reliable than sound(s), and thus if dwell time is one of the options, at block 304 electronic device 14 can be configured to determine—and some methods comprise determining—whether the patient can use the dwell time input. If yes, electronic device 14 can prioritize the dwell time input such that the sound(s) are not determined to be the icon selection input and, at block 308, electronic device 14 determines that the dwell time input is the icon selection input. This determination can be based on, for example, an assessment in which the patient practices using the dwell time input and/or an input from the user regarding whether the patient is capable of using the dwell time input.

If electronic device 14 at block 296 determines that the patient cannot make a facial gesture and—if the dwell time input is an option—at block 304 determines that the patient cannot use the dwell time input, then at block 312 the electronic device can be configured to determine and some methods comprise determining—that one of the sound(s) is the icon selection input. When there are multiple sounds to select from, electronic device 14 can be configured to determine which of the sounds is the icon selection input. Some sounds may be easier for the patient to make and/or for electronic device 14 to register. For example, in order of preference, the sounds can include tongue click, a pop sound, a “P” sound, a “K” sound, a “Ma” sound, and/or a “La” sound. Electronic device 14 can facilitate the selection of the sound that the patient can most-reliably use as the icon selection input. To illustrate, as shown in FIG. 16C, when determining which sound is the icon selection input, at blocks 362, 370, 378, 386, 394, and 402 electronic device 14 can be configured to successively determine—and some methods comprise successively determining-whether the patient can click their tongue, make a pop sound, make a “P” sound, make a “K” sound, make a “Ma” sound, and/or make a “La” sound until one of the determinations is yes, in which case the first of those sounds that the patient can make can, at one of blocks 366, 374, 382, 390, 398, or 406, respectively, be determined to be the icon selection input. The determination of which sounds the patient can make can be based at least in part on a sound assessment and/or an input from a user like the caregiver. If a sound assessment is used, electronic device 14 can be configured to test the patient's ability to make each of the sounds and the one that the electronic device can most-reliably detect can be determined to be the icon selection input. Additionally or alternatively, in some embodiments, a user such as a caregiver can input which of the sounds to use as the icon selection input (e.g., based on the caregiver's judgment), which can be a selection from a page displayed on electronic display 26 that includes the options (e.g., listed in the above-described order of preference). The device input can affect the selection too. For example, if the device input indicates that the patient is outfitted with a tracheostomy tube, electronic device 14 can be configured—and some methods can be performed-such that none of the “P” sound, “K” sound, “Ma” sound, and/or “La” sound are determined to be the icon selection input, as a patient so-outfitted cannot make those sounds. In such a case, if a sound assessment is performed, only tongue click and/or pop sound need be assessed, and if electronic device 14 permits a selection by the caregiver, the “P” sound, “K” sound, “Ma” sound, and/or “La” sound can be rendered unavailable as selectable options.

After the icon selection input is determined (e.g., to be a facial gesture, a dwell time input, or a sound), electronic device 14 can be configured to make the above-described visual acuity determination at block 284 to decide whether to use normal icons at block 288 or enlarged icons at block 292.

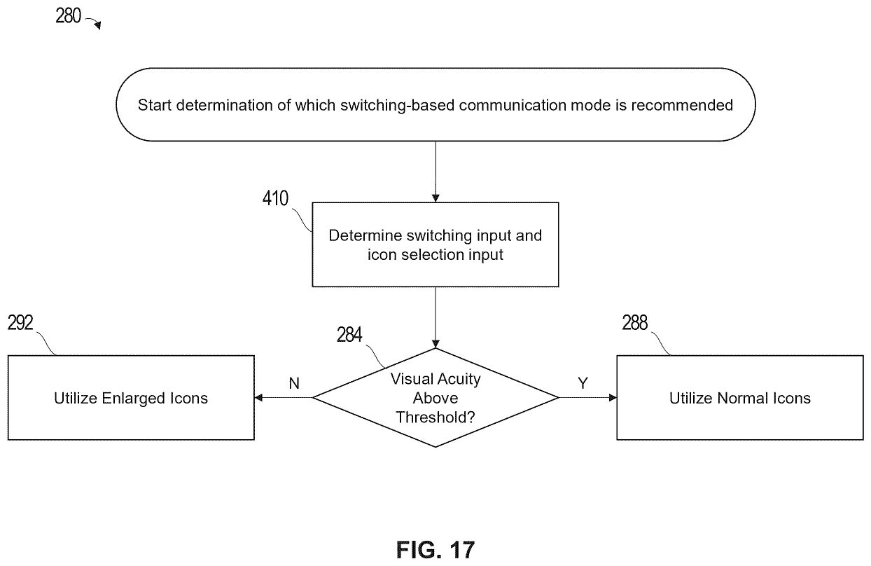

As also noted above, for each of the switching-based communication modes, the communication input can include both a switching input and an icon selection input that are different but that can each comprise a facial gesture and/or a sound. When, at block 280, electronic device 14 determines that a switching-based communication mode is the recommended communication mode (FIG. 14), as shown in FIG. 17, at block 410 the electronic device can be configured to determine—and some methods comprise determining—which of two or more types of inputs are the switching input and icon selection input. For a first one of the switching and icon selection inputs, the determination can be substantially the same as the above-described determination of the icon selection input for the head-based and eye-based communication modes: if electronic device 14 determines that the patient can make at least one of the facial gesture(s), one of the facial gesture(s) can be determined to be the first input in any of the manners described above (e.g., using the process of FIG. 16B), otherwise one of the sound(s) can be determined to be the first input in any of the manners described above (e.g., using the process of FIG. 16C). For a second one of the switching and icon selection inputs, the same can be done without selecting the type of input that was determined to be the first input. Thus, for example, if the patient can make only one of the facial gesture(s) and that facial gesture was determined to be the first input, then electronic device 14 can be configured to determine that one of the sound(s) is the second input. Additionally or alternatively, a user such as a caregiver can input which of the inputs (e.g., which of the facial gesture(s) and/or which of the sound(s)) to use as the switching and icon selection inputs.

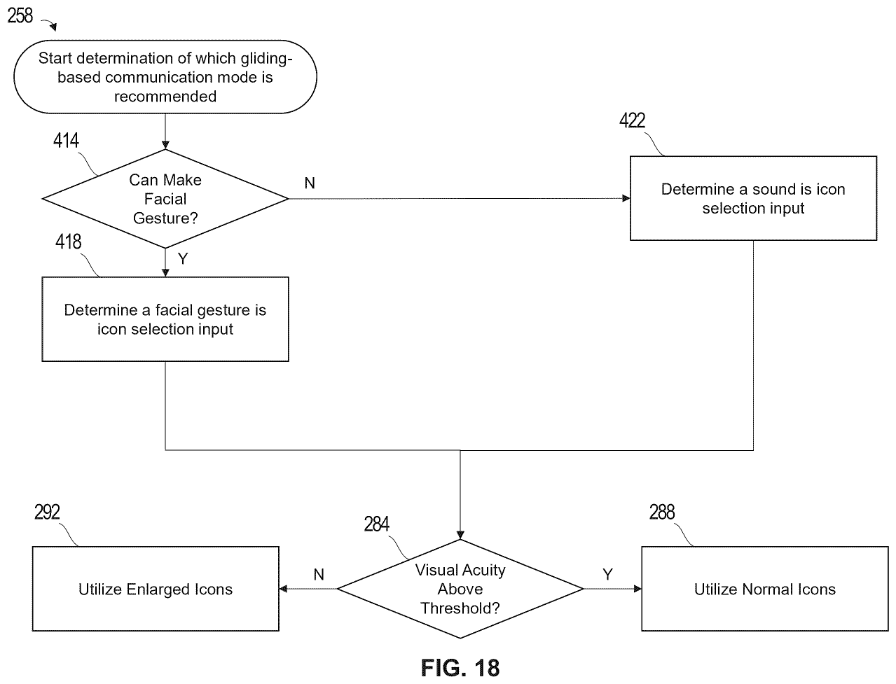

Referring to FIG. 18, when at block 258 electronic device 14 determines that one of the gliding-based communication mode(s) (e.g., one of the gliding-cursor-based communication mode(s) and/or one of the gliding-switch-based communication mode(s)) is the recommended communication mode, the electronic device can also be configured to determine—and some methods comprise determining—which of two or more types inputs (e.g., one or more facial gestures and/or one or more sounds) is the icon selection input and, if a gliding-cursor-based communication mode, the stop input. For the gliding-switch-based communication mode(s) in which only the icon selection input needs to be determined, this determination can also be substantially the same as the above-described determination of the icon selection input for the head-based and eye-based communication modes: as illustrated in FIG. 18, if at block 414 electronic device 14 determines that the patient can make at least one of the facial gesture(s), at block 418 the electronic device and be configured to determine—and some methods comprise determining—that one of the facial gesture(s) is the icon selection input in any of the manners described above (e.g., using the process of FIG. 16B), otherwise at block 422 one of the sound(s) can be determined to be the icon selection input in any of the manners described above (e.g., using the process of FIG. 16C). For the gliding-cursor-based communication mode(s) in which different stop and icon selection inputs need to be determined, the determination can be performed in the same way that the switching and icon selection inputs are determined for the switching-based communication mode(s).

With electronic device 14's determination of which of the communication modes is the recommended communication mode, including—where applicable—which of two or more types of inputs is the icon selection input, switching input, and/or stop input, the electronic device can facilitate its configuration in a way that accommodates the patient's abilities and allows the patient to more reliably interface with the electronic device to communicate with others by selecting icons 42.

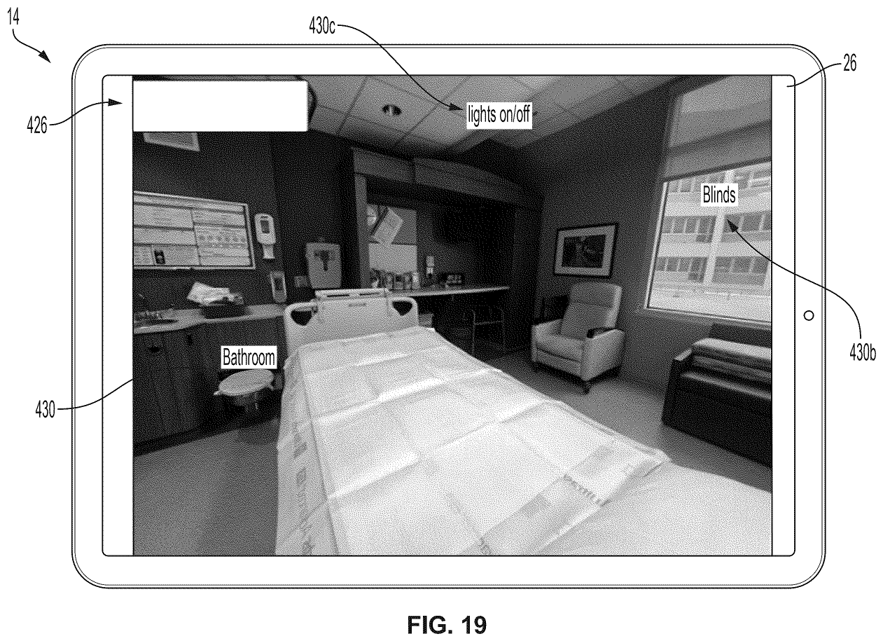

For some patients with significant mental impediments—such as those with traumatic brain injury (TBI)—it may be beneficial for the patient to use an interface that is simpler than communication page(s) 38a-38d to communicate with others. Referring to FIG. 19, in some embodiments electronic device 14 can have a scene-based mode in which the electronic device displays, on electronic display 26, a scene 426 that is representative of where the patient is located. For example, scene 426 can be a room with a healthcare facility that includes one or more selectable objects such as a toilet 430a, one or more blinds 430b, and/or one or more lights 430c. Electronic device 14 can be configured to determine which of the selectable object(s) is selected based at least in part on a communication input, such as a touch of one of the fingers of the patient at a location on electronic display 26 (e.g., touchscreen), where when the touched location overlies one of the selectable object(s), that selectable object is determined to be the selected object. Electronic device 14 can be configured to produce, via its speaker(s) 34, a sound based at least in part on which of the selectable object(s) is the selected object (e.g., a sound including words in the language that the caregiver can understand regarding what object is selected). This can allow the patient to communicate with others using contextualized objects that may be simpler for patients with, for example, TBI to understand.

### B. Medical Cart

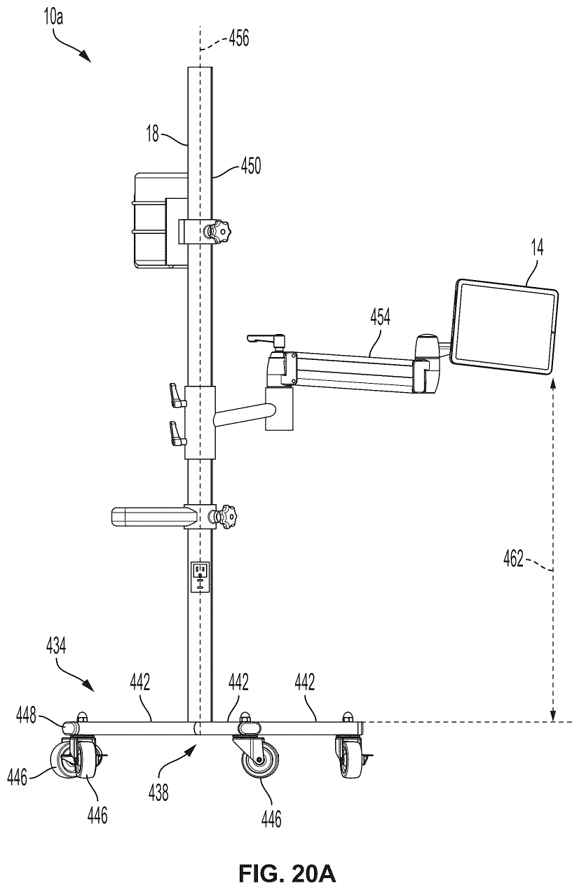

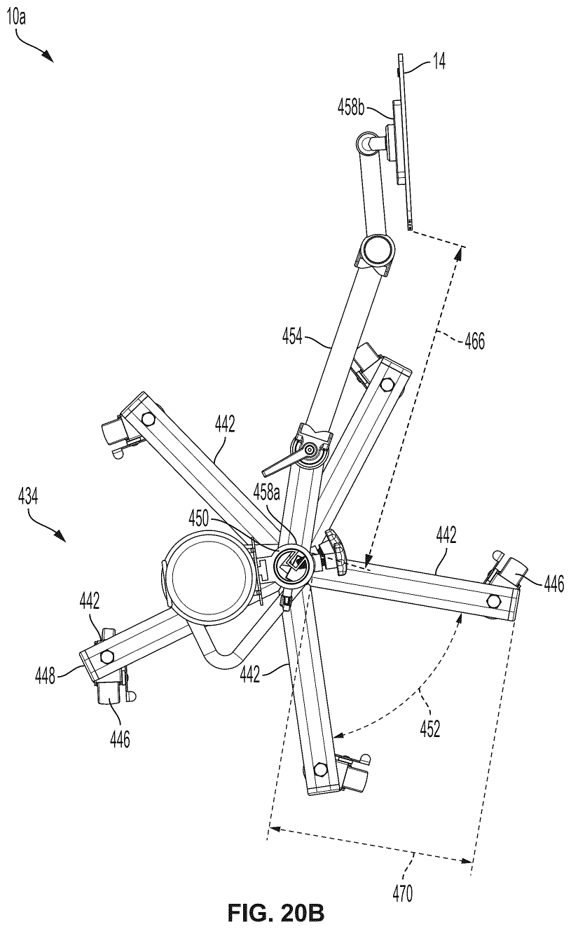

As explained above, system 10a can comprise a medical cart 18 to which electronic device 14 can be coupled, allowing the electronic device to be supported while the patient is using it. Referring again to FIG. 1 and also to FIGS. 20A and 20B, cart 18 can comprise a base 434 that includes a central portion 438, a plurality of supporting legs 442—such as greater than or equal to any one of, or between any two of, three four, five, six, seven, or eight supporting legs—that each extend at least radially outward from the central portion, and a plurality of wheels 446, each coupled to a respective one of the supporting legs such that the wheel is disposed closer to an outer end 448 of the supporting leg than to the central portion. Base 434's wheels 446 can accordingly be configured to be disposed a ground to allow cart 18 to stably roll on the ground. Wheels 446, as shown, each comprise a caster wheel to facilitate rolling in multiple directions. In some embodiments, cart 18 can comprise one or more motors configured to rotate at least one of wheels 446 to cause the cart to move, which can allow the cart to, for example, navigate (e.g., autonomously) to a target location (e.g., a bed holding a patient). To promote stability in multiple directions, for each of supporting legs 442, an angular separator 452, taken about a vertical axis 456 extending through central portion 438 of base 434, between the supporting leg and at least one—optionally two—other of the supporting legs can be less than or equal to any one of, or between any two of, 90°, 85°, 80°, 75°, 70°, 65°, 60°, 55°, 50°, 45°, 40°, 35°, or 30° (e.g., less than or equal to 80°).

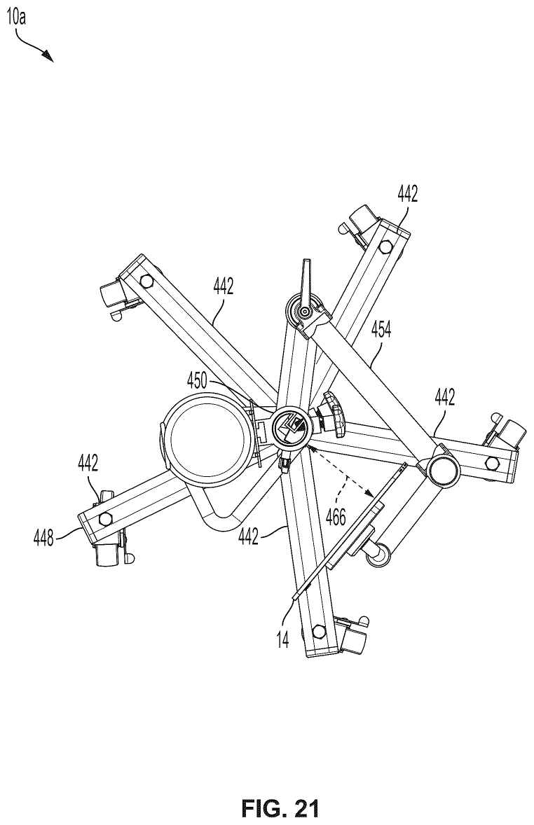

Cart 18 can also comprise a post 450 that is coupled to and extends upwardly away from central portion 438 of base 434 (e.g., such that vertical axis 456 extends through the post). Electronic device 14 can be coupled to post 450 via, for example, an arm 454 (e.g., with a first end 458a of the arm coupled to the post and a second end 458b of the arm coupled to the electronic device) such that the electronic device is disposed higher than base 434, which can allow the patient that can be above the ground (e.g., when standing, in a bed, in a chair, and/or the like) to have better access to the electronic device when using it. For example, a vertical distance 462 between electronic device 14 and base 434 can be greater than or equal to any one of, or between any two of, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 feet. Additionally, referring further to FIG. 21, electronic device 14 can be movable relative post 450 from a stowed position (FIG. 21) to a deployed position (FIG. 20B) (e.g., by moving arm 454) that is relatively far away from post 450 such that, for example, the electronic device can be positioned over a bed on which the patient is laying while base 434 is next to the bed, which can facilitate the patient's use of the electronic device. For example, when electronic device 14 is in the deployed position, a radial distance 466 between the electronic device and post 450 can be greater than or equal to any one of, or between any two of, 70%, 75%, 80%, 85%, 90%, 95%, 100%, 105%, 110%, 115%, or 120% (e.g., at least 80%) of radial distance 470 between central portion 438 of base 434 and outer end 448 of each of supporting legs 442. Radial distance 466 between electronic device 14 and post 450 can be smaller when in the stowed position than when in the deployed position to promote stability when cart 18 is rolled for transport.

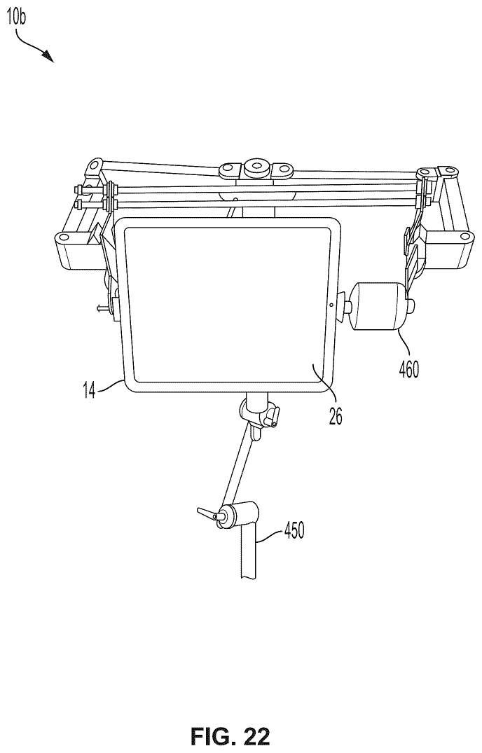

Referring further to FIG. 22—which shows a second embodiment 10b of the present systems that, except for the foregoing, is substantially the same as system 10a—in some embodiments cart 18 can comprise one or more motors 460 configured to move electronic device 14 relative to post 450. Electronic device 14 can be configured to control motor(s) 460 based at least in part on a position of a face of a patient that faces electronic display 26 (e.g., the touchscreen) of the electronic device, which can facilitate positioning of the electronic device in a way that promotes the patient's viewing of the electronic display and the electronic device's ability to register inputs from the patient (e.g., based on head movements, eye movements, facial gestures, and/or the like).

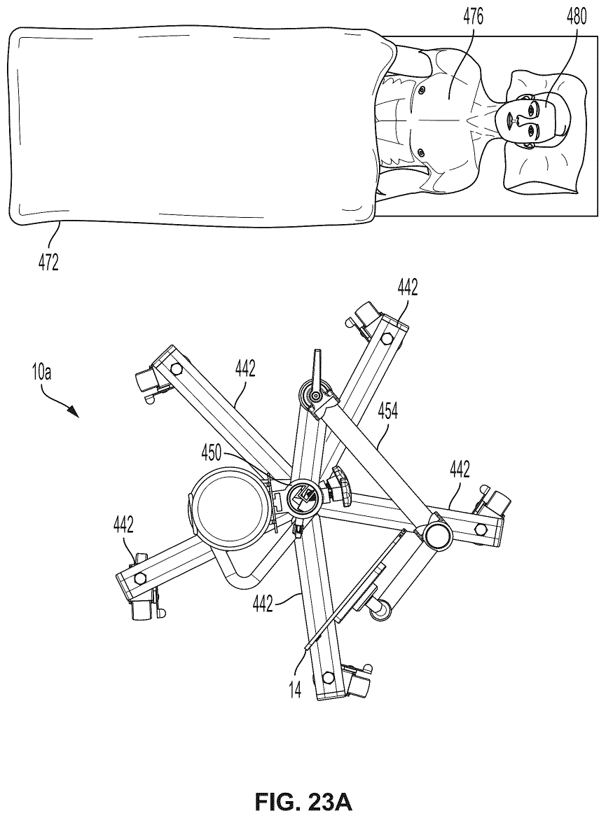

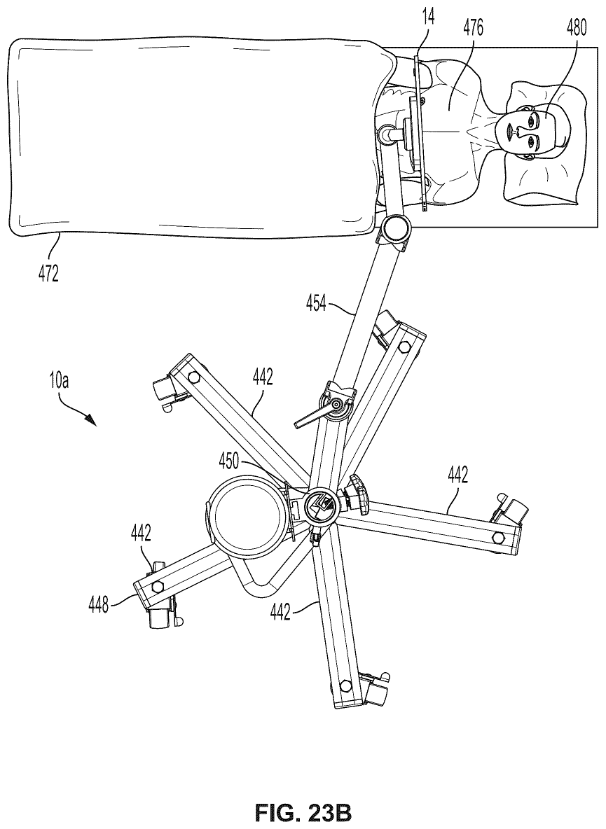

Referring to FIGS. 23A and 23B, some of the present methods of facilitating patient interactions comprise rolling a cart (e.g., 18) of a system (e.g., 10a or 10b) (e.g., any of those described above) that also includes an electronic device (e.g., 14) to a bed (e.g., 472) on which a patient (e.g., 476) is disposed (FIG. 23A). The patient can be disposed in a healthcare facility such as a hospital, and the cart can be rolled by a caregiver such as a nurse, a doctor, and/or the like or, if one or more of its wheels (e.g., 446) are powered (e.g., with one or more motors), the cart can roll itself to the bed. The electronic device can be in a stowed position when the cart is rolled to the bed, and some methods comprise moving the electronic device relative to a post (e.g., 450) of the cart to the deployed position (e.g., after the electronic device is at the bed) (FIG. 23B). Rolling the cart and moving the electronic device can be performed such that the electronic device is disposed closer to the patient than is the post. For example, as shown, after rolling the cart and moving the electronic device, the electronic device can be disposed over the bed and its electronic display (e.g., 26) can face a face (e.g., 480) of the patient, allowing the patient to readily use the electronic device in the manner explained above. One or more of the above-described assessments for determining which of the communication modes is the recommended communication mode (e.g., those that a caregiver performs) can, but need not, be performed before positioning the electronic device in this manner.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the apparatuses, systems, and methods are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A system for facilitating patient interactions, the system comprising:
an electronic device comprising:
one or more processors;
an electronic display; and
one or more speakers;
wherein the electronic device is configured to:
display, on the electronic display, one or more sensorimotor assessment pages that include a plurality of prompts, wherein each of the prompts requests a sensorimotor input that comprises a selection of one of a plurality options regarding a sensorimotor ability of a patient;
determine, based at least in part on the sensorimotor inputs, which of two or more communication modes of the electronic device is a recommended communication mode and:
automatically enter into the recommended communication mode; and/or
display, on the electronic display, which of the communication modes is the recommended communication mode;
display, on the electronic display, one or more communication pages, the communication page(s) including a plurality of icons that comprise a plurality of communication icons;
determine, based at least in part on a communication input, which of the icons of the communication page(s) is a selected icon, wherein for each of the communication modes, the communication input on which the determination is at least in part based is different when the electronic device is in the communication mode than when the electronic device is in at least one other of the communication modes; and
produce, via the speaker(s), a sound when the selected icon is one of the communication icons, the sound based at least in part on which of the communication icons is the selected icon.

2. The system of claim 1, wherein the communication modes of the electronic device comprise:
one or more cursor-based communication modes, wherein for each of the cursor-based communication mode(s), when the electronic device is in the cursor-based communication mode:
for each of the communication page(s), the electronic device is configured to display, on the electronic display, one or more cursors on the communication page; and
the communication input comprises an icon selection input that includes:
a facial gesture;
a sound; and/or
maintaining the cursor(s) within an area on one of the communication page(s) for greater than or equal to a threshold dwell time;
wherein the electronic device is configured such that, when the cursor(s) are disposed over one of the icons of the communication page(s) and the icon selection input is made, the electronic device determines that the icon over which the cursor(s) are disposed is the selected icon; and/or
one or more scroll-based communication modes, wherein for each of the scroll-based communication mode(s), when the electronic device is in the scroll-based communication mode:
the electronic device is configured to:
determine which one of the icons of the communication page(s) is a selectable icon; and
display, on the electronic display, an indication regarding which one of the icons of the communication page(s) is the selectable icon; and
the communication input comprises an icon selection input that includes:
a facial gesture; and/or
a sound;
wherein the electronic device is configured such that, for each of the icons of the communication page(s), when the icon is the selectable icon and the icon selection input is made, the electronic device determines that the icon is the selected icon.

3. The system of claim 2, wherein:
the communication modes of the electronic device comprise the cursor-based communication mode(s); and
the cursor-based communication mode(s) include:
one or more head-based communication modes, wherein for each of the head-based communication mode(s), when the electronic device is in the head-based communication mode, the communication input comprises movement of a head of the patient and, for each of the communication page(s), the electronic device is configured to determine a position of the cursor(s) on the communication page based at least in part on a position of the head of the patient; and/or
one or more eye-based communication modes, wherein for each of the eye-based communication mode(s), when the electronic device is in the eye-based communication mode, the communication input comprises movement of at least one of one or more eyes of the patient and, for each of the communication page(s), the electronic device is configured to determine a position of the cursor(s) on the communication page based at least in part on a position of at least one of the eye(s) of the patient.

4. The system of claim 3, wherein:
the cursor-based communication mode(s) include the head-based communication mode(s) and the eye-based communication mode(s); and
the prompts of the sensorimotor assessment page(s) comprise a head-movement prompt, wherein:
the sensorimotor input that the head-movement prompt requests comprises a selection of one of a plurality of options regarding whether the patient can move their head; and
the electronic device is configured such that when determining which of the communication modes is the recommended communication mode, when the sensorimotor input that the head-movement prompt requests indicates that the patient can move their head, none of the eye-based communication mode(s) are determined to be the recommended communication mode.

5. The system of claim 3, wherein:
the communication modes of the electronic device comprise the scroll-based communication mode(s); and
the prompts of the sensorimotor assessment page(s) comprise:
a head-movement prompt, wherein:
the sensorimotor input that the head-movement prompt requests comprises a selection of one of a plurality of options regarding whether the patient can move their head; and
the electronic device is configured such that when determining which of the communication modes is the recommended communication mode, when the sensorimotor input that the head-movement prompt requests indicates that the patient can move their head, none of the scroll-based communication mode(s) are determined to be the recommended communication mode; and/or
an eye-movement prompt, wherein:
the sensorimotor input that the eye-movement prompt requests comprises a selection of one of a plurality of options regarding whether the patient can move their eye(s); and
the electronic device is configured such that when determining which of the communication modes is the recommended communication mode, when the sensorimotor input that the eye-movement prompt requests indicates that the patient can move their eye(s) and the electronic device determines that it can track movement of the eye(s) of the patient and the patient is capable of moving the cursor(s) by moving their eye(s), one or more of the scroll-based communication mode(s) are not determined to be the recommended communication mode.

6. The system of claim 2, wherein:
the communication modes of the electronic device comprise the scroll-based communication mode(s); and
the scroll-based communication mode(s) comprise one or more switching-based communication modes, wherein for each of the switching-based communication mode(s), when the electronic device is in the switching-based communication mode:
the communication input comprises a switching input that includes:
a facial gesture; and/or
a sound;
wherein the switching input is different than the icon selection input; and
for each of the icons of the communication page(s), when the icon is the selectable icon and the switching input is made, the electronic device is configured to determine that the icon is no longer the selectable icon and that another one of the icons is the selectable icon.

7. The system of claim 2, wherein:
the communication modes of the electronic device comprise the cursor-based communication mode(s); and
the cursor-based communication mode(s) comprise one or more gliding-cursor-based communication modes, wherein for each of the gliding-cursor-based communication mode(s), when the electronic device is in the gliding-cursor-based communication mode:
the one or more cursors comprise first and second linear cursors, the first linear cursor extending in a first direction and the second linear cursor extending in a second direction that is substantially perpendicular to the first direction;
the communication input comprises a stop input that includes:
a facial gesture; and/or
a sound;
for each of the communication page(s), the electronic device is configured to display, on the electronic display:
the first linear cursor moving in the second direction, wherein the electronic device is configured to stop the first linear cursor and display the first linear cursor at a position at which the first linear cursor is stopped when the stop input is made; and
after the first linear cursor is stopped, the second linear cursor moving in the first direction;
wherein when an intersection point at which the first linear cursor intersects the second linear cursor is disposed over one of the icons of the communication page(s) and the icon selection input is made, the electronic device is configured to determine that the icon over which the intersection point is disposed is the selected icon.

8. The system of claim 7, wherein:
the electronic device is configured to display, on the electronic display, one or more consciousness assessment pages that include a prompt that requests a consciousness input, wherein:
the consciousness input comprises a selection of one of a plurality of options regarding a level of consciousness of the patient; and
the options include a first level of consciousness and a second level of consciousness that is lower than the first level of consciousness; and
the electronic device is configured such that when determining which of the communication modes is the recommended communication mode, when the consciousness input indicates that the patient has the second level of consciousness, one of the gliding-cursor-based communication mode(s) is determined to be the recommended communication mode.

9. The system of claim 2, wherein:
the communication modes of the electronic device comprise the scroll-based communication mode(s); and
the scroll-based communication mode(s) comprise one or more gliding-switch-based communication modes, wherein for each of the gliding-switch-based communication mode(s), when the electronic device is in the gliding-switch-based communication mode:
for each of the icons of the communication page(s), the electronic device is configured such that when the icon has been the selectable icon for a threshold selection time, the electronic device determines that the icon is no longer the selectable icon and that another one of the icons is the selectable icon.

10. The system of claim 9, wherein:
the electronic device is configured to display, on the electronic display, one or more consciousness assessment pages that include a prompt that requests a consciousness input, wherein:
the consciousness input comprises a selection of one of a plurality of options regarding a level of consciousness of the patient; and
the options including a first level of consciousness and a second level of consciousness that is lower than the first level of consciousness; and
the electronic device is configured such that when determining which of the communication modes is the recommended communication mode, when the consciousness input indicates that the patient has the second level of consciousness, one of the gliding-switch-based communication mode(s) is determined to be the recommended communication mode.

11. The system of claim 2, wherein:
the electronic device is configured to determine whether the patient is capable of making at least one of one or more facial gestures; and
for each of the cursor-based communication mode(s) and/or scroll-based communication mode(s):
the electronic device is configured to determine which one of two or more types of selection inputs is the icon selection input, the types of selection inputs including:
one or more facial gestures; and
one or more sounds; and
the electronic device is configured such that when determining which of the types of selection inputs is the icon selection input, when the electronic device determines that the patient is capable of making at least one of one or more facial gestures, none of the sound(s) of the types of selection inputs are determined to be the icon selection input.

12. The system of claim 2, wherein:
the electronic device is configured to determine whether the patient is capable of making:
one or more lower facial gestures that are each from a portion of a face of the patient that is below a nose of the patient; and
one or more upper facial gestures that are each from a portion of the face of the patient that is at or above the nose of the patient; and
for each of the cursor-based communication mode(s) and/or scroll-based communication mode(s):
the electronic device is configured to determine which one of two or more types of selection inputs is the icon selection input, the types of selection inputs including:
the lower facial gesture(s); and
the upper facial gesture(s); and
the electronic device is configured such that when determining which of the types of selection inputs is the icon selection input, when the electronic device determines that the patient is capable of making at least one of the lower facial gesture(s), none of the upper facial gesture(s) are determined to be the icon selection input.

13. The system of claim 12, wherein the lower facial gesture(s) comprise:
poking out a tongue;
opening a mouth;
smiling; and/or
grimacing.

14. The system of claim 1, wherein:
the electronic display of the electronic device comprises a touchscreen;
the communication modes of the electronic device comprise one or more touch-based communication modes, wherein for each of the touch-based communication mode(s), when the electronic device is in the touch-based communication mode:
the communication input comprises an icon selection input that includes touching the touchscreen;
wherein the electronic device is configured such that when the touchscreen is touched at a location disposed over one of the icons of the communication page(s), the electronic device determines that the icon over which the touched location is disposed is the selected icon.

15. The system of claim 14, wherein
the prompts of the sensorimotor assessment page(s) comprise a touch prompt, wherein the sensorimotor input that the touch prompt requests comprises a selection of one of a plurality of options regarding whether the patient can touch the touchscreen with one of a plurality of fingers of the patient; and
the electronic device is configured such that when determining which of the communication modes is the recommended communication mode, when the sensorimotor input that the touch prompt requests indicates that the patient can touch the touchscreen with one of the fingers of the patient, one of the touch-based communication mode(s) is determined to be the recommended communication mode.

16. The system of claim 1, wherein the electronic device is configured to:
display, on the electronic display, one or more device assessment pages that include a prompt that requests a device input, the device input comprising a selection of at least one of a plurality of options regarding whether the patient is outfitted with one or more devices; and
for at least one of the sensorimotor inputs, preselect, based at least in part on the device input, one of the options regarding the sensorimotor ability of the patient.

17. The system of claim 16, wherein:
the prompts of the sensorimotor assessment page(s) comprise:
a head-movement prompt, wherein the sensorimotor input that the head-movement prompt requests comprises a selection of one of a plurality of options regarding whether the patient can move their head;
a facial-gesture prompt, wherein the sensorimotor input that the facial-gesture prompt requests comprises a selection of one of a plurality of options regarding whether the patient can make a facial gesture; and
a sound prompt, wherein the sensorimotor input that the sound prompt requests comprises a selection of one of a plurality options regarding whether the patient can make a sound with their mouth and/or tongue;
the one or more devices comprise:
one or more immobility devices configured to inhibit movement of the head of the patient, wherein the electronic device is configured such that when the device input indicates that the patient is outfitted with at least one of the immobility device(s), the electronic device preselects one of the options for the head-movement prompt indicating that the patient cannot move their head; and
one or more mouth devices configured to be disposed on and/or through the mouth of the patient, wherein the electronic device is configured such that when the device input indicates that the patient is outfitted with at least one of the mouth device(s), the electronic device preselects one of the options for the facial-gesture prompt indicating that the patient cannot make a facial gesture and one of the options for the sound prompt indicating that the patient cannot make a sound with their mouth and/or tongue.

18. The system of claim 1, wherein:
the prompts of the sensorimotor assessment page(s) comprise a visual-acuity prompt, wherein:
the sensorimotor input that the visual-acuity prompt requests comprises a selection of one of a plurality of options regarding a visual acuity of the patient; and
the options of the sensorimotor input of the visual-acuity prompt include a first level of visual acuity and a second level of visual acuity that is lower than first level of visual acuity;
for each of the communication modes of a first portion of the communication modes of the electronic device, when the electronic device is in the communication mode, each of the communication icons has a first area;
for each of the communication modes of a second portion of the communication modes of the electronic device, when the electronic device is in the communication mode, each of the communication icons has a second area that is larger than the first area; and
the electronic device is configured to determine that one of the communication modes of the second portion of the communication modes is the recommended communication mode when the sensorimotor input that the visual-acuity prompt requests indicates that the patient has the second level of visual acuity.

19. The system of claim 1, wherein:
the electronic device is configured to display, on the electronic display, one or more language assessment pages that include:
a caregiver language prompt that requests a caregiver language input that comprises a selection of one of a plurality of options regarding a language that a caregiver is able to understand; and
a patient language prompt that requests a patient language input that comprises a selection of one of a plurality of options regarding a language that the patient is able to understand;
each of the communication icons includes a marking in the language that the patient language input indicates the patient is able to understand; and
the sound that the electronic device is configured to produce based at least in part on which of the communication icons is the selected icon includes a sound in the language that the caregiver language input indicates the caregiver is able to understand.

20. The system of claim 1, comprising a medical cart that includes:
a base that includes:
a central portion;
a plurality of supporting legs that each extend at least radially outward from the central portion; and
a plurality of wheels, each coupled to a respective one of the supporting legs such that the wheel is disposed closer to an outer end of the supporting leg than to the central portion; and
a post that is coupled to and extends upwardly away from the central portion of the base;
wherein the electronic device is coupled to the post such that the electronic device is disposed higher than the base.
